(19) United States
(12) Patent Application Publication
(10) Pub. No.: US 2024/0026438 A1
(43) Pub. Date: Jan. 25, 2024

(54) DETECTION OF NUCLEIC ACIDS USING DIRECT RT-PCR FROM BIOLOGICAL SAMPLES

(71) Applicant: DIANA BIOTECHNOLOGIES, S.R.O., Vestec (CZ)

(72) Inventor: Václav NAVRÁTIL, Liberec (CZ)

(21) Appl. No.: 18/256,514

(22) PCT Filed: Dec. 9, 2021
(86) PCT No.: PCT/IB2021/061528

§ 371 Date: Jun. 8, 2023

Related U.S. Application Data
(60) Provisional application No. 63/123,323, filed on Dec. 9, 2020.

(51) Int. Cl. C12Q 1/686

(52) U.S. Cl. CPC C12Q 1/686

(57) ABSTRACT

The present disclosure generally relates to systems, compositions, kits, and methods for detection of nucleic acids in biological samples. The present disclosure also relates to ultrasensitive direct detection of pathogenic nucleic acids in various biological samples without the need to isolate the nucleic acids from the samples. The present disclosure further relates to detection of airborne or blood borne viruses directly from biological samples.

### CROSS-REFERENCE TO RELATED APPLICATIONS

This application claims the benefit of U.S. Provisional Patent Application No. 63/123,323, filed on Dec. 9, 2020, and entitled “Detection Of Nucleic Acids Using Direct RT-PCR From Biological Samples,” the entirety of which is incorporated by reference.

### FIELD OF THE DISCLOSURE

The present disclosure generally relates to systems, compositions, kits, and methods for detection of nucleic acids in biological samples. The present disclosure also relates to ultrasensitive direct detection of pathogenic nucleic acids in various biological samples without the need to isolate the nucleic acids from the samples. The present disclosure further relates to detection of airborne or blood borne viruses directly from biological samples.

### BACKGROUND

Many diseases are diagnosed by polymerase chain reaction (PCR), such as reverse transcription quantitative PCR (RT-qPCR), in particular respiratory viruses such as Influenza and SARS, or blood borne viruses such as Hepatitis and HIV. PCR or RT-qPCR is also used to detect pathogenic bacterial nucleic acids, e.g. of sexually transmitted pathogens in urogenital tract. These methods can be used also to detect human RNA or DNA to detect changes in expression of human genes or to detect genetic alterations in order to detect genetic diseases or oncology diseases. The test samples can be various biological specimens, most frequently swab transport media, saliva, or blood, including whole blood, serum, or plasma. To detect the pathogen, it is necessary to purify the target nucleic acids (including at least one of DNA or RNA) from the biological sample. Currently, it is not possible to add the sample directly to the RT-qPCR reaction because the sample inhibit the reaction and/or degrade the target nucleic acid during the reaction. Unfortunately, the nucleic acid extraction methods, most frequently relying either on silica coated magnetic beads or on silica spin columns, are complicated and time consuming and result in loss of the genetic material during the process.

During the SARS-Cov-2 (COVID-19) pandemics, there is an urgent need to develop more efficient nucleic acid extraction methods or even to develop methods without nucleic acid extraction. Such method would not only make the process faster and cheaper, but it could also increase the sensitivity and enable for higher throughput. It could also make the process safer to the lab workers, if the samples could be inactivated without manual handling of the samples.

There has been significant effort to develop such methods. For example, LabCorp® developed a heat RNA extraction method for which it received FDA emergency use authorization. Further, University of Illinois developed heat treatment of saliva samples followed by dilution in buffer formulated to lyse the virus followed by standard RT-qPCR. In this instance, the saliva must be diluted in a Tris/Borate/EDTA (TBE) buffer, followed by heat inactivated for at least 30 minutes at 95° C. In this method, shorter time or lower temperature leads to loss of sensitivity. Next, the process requires detergents to be added and followed by a regular RT-PCR with conventional RT-PCR buffers. As can be seen, the process is complicated and, even at its optimum, leads to higher cycle threshold (Ct) values compared to RT-PCR after conventional RNA extraction.

Moreover, University of Yale developed a “SalivaDirect™” protocol, which treats the samples in other way. Briefly, proteinase K is added, followed by vortexing the samples and heat inactivation at 95° C. In the Yale process, conventional RT-PCR mixtures are used for detection. However, only some of the commercially available kits are suitable, and there is no information on why this is the case. Additionally, the process includes handling of native viscous and infectious saliva.

Thus, all of these available methods rely on complex sample pretreatment, which leads to dilution of the samples and sensitivity loss. Further, these processes sometimes require pipetting of viscous and infectious saliva. Moreover, these pretreatment methods only work with some RT-PCR kits and not others. The developers give no hint why this is the case and whether it would be possible to redesign the RT-PCR kits in order to skip these pretreatment steps and detect the pathogens, such as the SARS-Cov-2 virus, by just adding the sample into the RT-PCR mix. Currently, this is not possible as these references clearly shows that even the best RT-PCR methods are not suitable for direct detection of the virus.

### SUMMARY

As a solution to the issues in the field, embodiments of the present disclosure provide novel systems and methods for PCR, such as direct RT-PCR, which offer easy and safe detection of nucleic acids directly from biological samples without the need of nucleic acid extraction steps or sample pretreatment. Moreover, the systems and methods provided herein are compatible with pretreated samples, such as heat inactivation or proteinase K treatment, which are commonly used in the field.

In an embodiment, a system for reverse transcriptase polymerase chain reaction (RT-PCR) is provided. The system can include a buffer, a salt, a mixture of deoxynucleotide triphosphates (dNTPs), a detergent, a reducing agent, an RNA carrier, a thermostable DNA polymerase, a reverse transcriptase, and an RNAse inhibitor.

In another embodiment, the buffer can include at least one of Tris, Bis-tris-propane, PIPES, MOPS, or HEPES.

In another embodiment, the salt can include at least one salt. The at least one salt can include potassium chloride, ammonium sulfate, magnesium chloride, or magnesium sulfate, alone or in any combination.

In another embodiment, the buffer can include glycerol.

In another embodiment, the buffer can include dimethyl sulfoxide (DMSO).

In another embodiment, the buffer can include Bovine serum albumin (BSA) or casein.

In another embodiment, the thermostable DNA polymerase can include at least one of a Taq polymerase, a Tth Polymerase, a Bst polymerase, or a Z05 polymerase, alone or in any combination.

In another embodiment, the thermostable DNA polymerase is a wild-type enzyme.

In another embodiment, the thermostable DNA polymerase can include one or more single point mutations or is on N-terminus, C-terminus, internally truncated, or fused to another peptide or protein.

In another embodiment, the RT-PCR can include at least one of a quantitative reverse transcription PCR (RT-qPCR), a loop-mediated isothermal amplification (LAMP), a RT-LAMP, or any combination thereof.

In another embodiment, the thermostable DNA polymerase does not have the 5′-3′ exonuclease activity.

In another embodiment, the thermostable DNA polymerase has 5′-3′ exonuclease activity.

In another embodiment, the buffer can include Tris at a concentration within the range from about 10 to about 100 mM.

In another embodiment, the salt can include potassium chloride at a concentration within the range from about 50 to about 100 mM.

In another embodiment, the salt can include magnesium chloride at a concentration within the range from about 2 to about 5 mM.

In another embodiment, the salt can include ammonium sulfate at a concentration within the range from about 20 to about 50 mM.

In another embodiment, the salt can include magnesium sulfate at a concentration within the range from about 1 to about 5 mM.

In another embodiment, the mixture of dNTPs can include dATP, dCTP, dTTP, and dGTP, each at a concentration within the range from about 0.05 to about 0.5 mM.

In another embodiment, the mixture of dNTPs can include dATP, dCTP, dUTP, and dGTP, each at a concentration within the range from about 0.05 to about 0.5 mM.

In another embodiment, the reverse transcriptase is thermostable.

In another embodiment, the reverse transcriptase can include M-MLV, AMV, or FeLV reverse transcriptase.

In another embodiment, the reverse transcriptase is a wild-type enzyme.

In another embodiment, the reverse transcriptase can include one or more single point mutations or is on N-terminus, C-terminus, or internally truncated or fused to another peptide or protein.

In another embodiment, the reverse transcriptase is an RNAse H− mutant.

In another embodiment, the reverse transcriptase is inactivated by aptamer-oligonucleotides at about room temperature (e.g., warm-started).

In another embodiment, the reverse transcriptase is inactivated by aptamer-oligonucleotides at temperatures of up to about 45° C.

In another embodiment, the concentration of the reverse transcriptase is higher than about 0.5 U/uL.

In another embodiment, the concentration of the reverse transcriptase is within the range from about 0.05 to about 0.5 U/uL.

In another embodiment, the concentration of the DNA polymerase is higher than about 2 U/uL.

In another embodiment, the concentration of the DNA polymerase is within the range from about 0.02 to about 2 U/uL.

In another embodiment, the DNA polymerase is inactivated by aptamer-oligonucleotides, anti-DNA polymerase antibodies, or chemical modifications at about room temperature (e.g., hot-started).

In another embodiment, the DNA polymerase is inactivated by aptamer-oligonucleotides at temperatures of up to about 55° C.

In some embodiments, the reducing agent is selected from the list consisting of Dithiothreitol (DTT), β-mercaptoethanol, tris(2-carboxyethyl)phosphine (TCEP), glutathione, acetyl L-cystein, acetyl D-cystein, L-Cysteine methyl ester, D-Cysteine methyl ester, L-Cysteine methyl ester, D-Cysteine methyl ester, N-Formyl-L-cysteine, Tris(hydroxypropyl)phosphine, Tris(hydroxymethyl)phosphine, Sodium triacetoxyborohydride, 1,2-Ethanedithiol, 2-Merc aptopropan-1-ol, 3-Merc aptopropan-1-ol, 1-mercaptopropan-2-ol, Thioglycolic acid and a salt, Dithiothreitol, 2-Mercaptobenzoic acid, 3-Mercaptobenzoic acid, 4-Mercaptobenzoic acid, 4-Mercaptobutan-1-ol, Cysteamine, homocysteine, N-Acetyl-L-homocysteine, L-homocysteine methyl ester, 3-mercaptobutanol, Dihydrolipoic acid, dithiobutylamine, sodium sulfite, NADH, FADH₂, 2,3-Pyrazinedithiol, thiourea, or thiolactic acid.

In another embodiment, the reducing agent is Dithiothreitol (DTT).

In another embodiment, the concentration of DTT is higher than about 0.01 mM.

In another embodiment, the concentration of DTT is within the range from about 0.1 to about 1.0 mM.

In another embodiment, the RNAse inhibitor remains active at a temperature up to at least about 40° C.

In another embodiment, the RNAse inhibitor is selected from the list consisting of a porcine liver RNAse inhibitor, a human placental RNAse inhibitor, a murine RNAse inhibitor, a rat lung RNAse inhibitor, or a rat liver RNAse inhibitor.

In another embodiment, the RNAse inhibitor can include one or more single point mutations or is on N-terminus, C-terminus or internally truncated or fused to another peptide or protein.

In another embodiment, the concentration of RNAse inhibitor is about 0.1 U/uL or higher.

In another embodiment, the concentration of RNAse inhibitor is within the range of about 0.01 to 0.1 U/uL.

In another embodiment, the concentration of the RNA carrier is within the range from about 0.0005 to about 0.05 mg/mL.

In another embodiment, the concentration of the RNA carrier is within the range from about 0.002 to 0.01 mg/mL.

In another embodiment, the RNA carrier can be a polyinosinic acid, a polyinosinic-polycytidylic acid, or a polyadenosine.

In another embodiment, the RNA carrier comprises a polyinosinic acid.

In another embodiment, the RNA carrier comprises a polyadenosine.

In another embodiment, the system has a pH within the range of about 8.2 to about 8.8.

In another embodiment, the system has a pH within the range of about 8.4 to about 8.6.

In another embodiments, the detergent is a nonionic detergent.

In another embodiment, the concentration of the nonionic detergent is within the range from about 0.05% to about 5%.

In another embodiment, the concentration of the nonionic detergent is within the range from about 0.1% to about 2.0%.

In another embodiment, the concentration of the nonionic detergent is within the range from about 0.2% to about 1.0%.

In another embodiment, the concentration of the nonionic detergent is about 0.5%.

In another embodiment, the nonionic detergent can be at least one of Tween 20, Tween 40, Tween 80, NP-40, Triton™ X-100, C₁₂E₈, or dodecylmaltoside (DDM).

In another embodiment, the system can include one or more primers. In another embodiment, the one or more primers are configured to hybridize to a target nucleic acid. In another embodiment, the target nucleic acid is derived from a biological sample. In another embodiments, the target nucleic acid is detected directly in the biological sample without extracting it from the sample.

In another embodiments, the target nucleic acid can include at least one of RNA or DNA.

In another embodiment, the target nucleic acid can include a viral RNA or viral DNA, bacterial RNA or bacterial DNA, animal RNA or animal DNA, human RNA or human DNA.

In another embodiment, the target nucleic acid is SARS-Cov-2 RNA, SARS-1 (2003), MERS, influenza A, influenza B, respiratory syncytial virus (RSV), or other respiratory viruses, Hepatitis A, Hepatitis B, Hepatitis C, or HIV.

In some embodiments, the system can include one or more primers and/or one or more dual labeled probes targeting a region in at least one of a SARS-CoV-2 EndoRNAse gene, a SARS-CoV-2 Spike gene, a SARS-CoV-20RF1ab gene, a SARS-CoV-2 RdRP gene, a viral E gene, or a SARS-CoV-2 N gene.

In another embodiment, the system can include a primer pair and dual labeled probes configured to target a region in a viral EndoRNAse gene and a viral Spike gene, respectively.

In another embodiment, the system can further include one or more primers and/or one or more dual labeled probes configured to target a region in an internal human control gene.

In another embodiment, the system can further include one or more primers and/or one or more dual labeled probe configured to target an external RNA or DNA control.

In another embodiment, the system can further include one or more primers and/or one or more dual labeled probes configured to target a region in the SARS-CoV-2 EndoRNAse gene, the SARS-CoV-2 Spike gene, and the human RNAse P gene. The SARS-CoV-2 EndoRNAse gene can be labeled with FAM, the SARS-CoV-2 Spike gene can be labeled with HEX, and the human RNAse P gene can be labeled with Cy5.

In another embodiment, the system can further include one or more primers and/or one or more dual labeled probes configured to target a region in a SARS-CoV-2 EndoRNAse gene, a SARS-CoV-2 Spike gene, and an external artificial RNA control. The SARS-CoV-2 EndoRNAse gene can be labeled with FAM, the SARS-CoV-2 Spike gene can be labeled with HEX, and the external artificial RNA control can be labeled with Cy5.

In another embodiment, the system can further include one or more primers and/or one or more dual labeled probes configured to target a region in a SARS-CoV-2 EndoRNAse gene, a SARS-CoV-2 Spike gene, a human RNAse P gene, and an external artificial RNA control. The SARS-CoV-2 EndoRNAse gene can be labeled with FAM, the SARS-CoV-2 Spike gene can be labeled with HEX, the human RNAse P gene can be labeled with Texas Red, and the external control can be labeled with Cy5.

In another embodiment, the system can further include one or more primers and/or one or more dual labeled probes configured to target a region in a SARS-CoV-2 EndoRNAse gene, a SARS-CoV-2 Spike gene, and an external artificial RNA control. The SARS-CoV-2 EndoRNAse gene can be labeled with FAM, the SARS-CoV-2 Spike gene can be labeled with FAM, and the external artificial RNA control can be labeled with HEX.

In another embodiment, the system can further include one or more primers and/or one or more dual labeled probes configured to target a region in a SARS-CoV-2 EndoRNAse gene, a SARS-CoV-2 Spike gene, and an external artificial RNA control. The SARS-CoV-2 EndoRNAse gene can be labeled with FAM, the SARS-CoV-2 Spike gene can be labeled with FAM, and the human RNAse P control can be labeled with HEX.

In another embodiment, the system can further include one or more primers and/or one or more dual labeled probes configured to target a region in a SARS-Cov-2 EndoRNAse gene, a SARS-CoV-2 Spike gene, an Influenza A genome, an Influenza B genome, and a human RNAse P gene. The SARS-CoV-2 EndoRNAse gene can be labeled with FAM, the SARS-CoV-2 Spike gene can be labeled with FAM, the Influenza A genome can be labeled with HEX, the Influenza B genome can be labeled with Texas Red, and the human RNAse P gene can be labeled with Cy5.

In another embodiment, the system can further include one or more primers and/or one or more dual labeled probes configured to target a region in a SARS-Cov-2 EndoRNAse gene, a SARS-CoV-2 Spike gene, an Influenza A genome, an Influenza B genome, and an external artificial RNA control. The SARS-CoV-2 EndoRNAse gene can be labeled with FAM, the SARS-CoV-2 Spike gene can be labeled with FAM, the Influenza A genome can be labeled with HEX, the Influenza B genome can be labeled with Texas Red, and the external artificial RNA control can be labeled with Cy5. In another embodiment, the system can further include one or more primers and/or one or more dual labeled probes configured to target a region in a SARS-Cov-2 EndoRNAse gene, a SARS-CoV-2 Spike gene, an Influenza A genome, an Influenza B genome, an RSV A genome, an RSV B genome, and a human RNAse P gene, wherein the SARS-CoV-2 EndoRNAse gene can be labeled with FAM, the SARS-CoV-2 Spike gene can be labeled with FAM, the Influenza A genome can be labeled with HEX, the Influenza B genome can be labeled with Texas Red, the RSV A genome can be labeled with Cy5.5, the RSV B genome can be labeled with Cy5.5, and the human RNAse P gene can be labeled with Cy5.

In another embodiment, the system can further include one or more primers and/or one or more dual labeled probes configured to target a region in a SARS-Cov-2 EndoRNAse gene, a SARS-CoV-2 Spike gene, an Influenza A genome, an Influenza B genome, an RSV A genome, an RSV B genome, and an external artificial RNA control, wherein the SARS-CoV-2 EndoRNAse gene can be labeled with FAM, the SARS-CoV-2 Spike gene can be labeled with FAM, the Influenza A genome can be labeled with HEX, the Influenza B genome can be labeled with Texas Red, the RSV A genome can be labeled with Cy5.5, the RSV B genome can be labeled with Cy5.5, and the external artificial RNA control can be labeled with Cy5.

In an embodiment, the present disclosure also provides a kit including the system described herein.

In another embodiment, the kit can further include one or more control samples, PCR grade water, or combinations thereof.

In another embodiment, the one or more control samples can include a positive control sample, a negative control sample, or both.

In another embodiments, the one or more control samples can include an external RNA control.

In another embodiment, the kit can further include an instruction.

In an embodiment, a method for detecting a target nucleic acid derived from a biological sample is provided. In some embodiments, the method can include contacting the biological sample with the system described herein. In some embodiments, the method can further include subjecting the biological sample and the system to RT-PCR.

In some embodiments, the RT-PCR can include at least one of quantitative reverse transcription PCR (RT-qPCR), a reverse transcription loop-mediated isothermal amplification (RT-LAMP), a LAMP or any combination thereof.

In another embodiment, the method does not include extracting the target nucleic acid from the biological sample.

In another embodiment, the method does not include pretreatment of the biological sample.

In another embodiment, the biological sample is not been pretreated.

In another embodiment, the biological sample is pretreated.

In another embodiments, the biological sample is pretreated with at least one of heat or proteinase K. In other embodiments, the biological sample is pretreated with both heat and proteinase K.

In another embodiment, the biological sample is pretreated by heating to a temperature within the range from about 65° C. to about 95° C. for about 10 to about 60 minutes.

In another embodiment, the biological sample is centrifuged.

In another embodiment, the biological sample is a pooled sample including target nucleic acids from multiple subjects.

In another embodiment, the target nucleic acids from the multiple subjects are detected in one reaction.

In another embodiment, the biological sample can include at least one of blood, blood serum, blood plasma (anticoagulated with EDTA or heparin or citrate), saliva, nasal swab, nasopharyngeal swab, nasal wash, mouth swab, mouth wash, seminal plasma, or urine or any combination thereof.

In another embodiments, the target nucleic acid can include at least one of DNA or RNA.

In another embodiment, the method further includes quantifying the target nucleic acid amplified by the RT-PCR.

### BRIEF DESCRIPTION OF THE DRAWINGS

FIG. 1 shows the sensitivity of SARS-Cov-2 determined with serial dilutions of viral RNA detection by the direct RT-PCR method provided in the present disclosure. Detection of each concentration was done in 12-replicates and each replicate was positive even for the lowest tested amount of 10 copies per reaction. The copy number stands for the total number of copies in the reaction. Five μL of the sample were added to the reaction, the copy number per μL, of the sample is thus 5-fold lower. That is, each replicate was positive even for the lowest tested amount of 2 copies per μL. The amount of viral RNA was quantified against standard with known concentration. The Y-axis shows the Ct values for detection of viral gene EndoRNAse.

FIGS. 2A-2F show the detection of SARS-Cov-2 virus spiked into various matrices: buffer (FIG. 2A), pooled human blood serum (FIG. 2B), Copan universal transport medium (FIG. 2C), untreated human pooled saliva (FIG. 2D), heat inactivated human pooled saliva, at 65° C. (FIG. 2E) and 80° C. (FIG. 2F) for 10 minutes. Serial dilution of virus was detected in each matrix: 100,000; 10,000; 1,000; 100; 50; and 20 copies per reaction (copies per μL of the sample were 5-fold lower, that is 20,000; 2,000, 200; 20; 10 and 4 copies per μL). Threshold cycles for detection of viral EndoRNAse are shown.

FIGS. 3A-3C show the direct detection of SARS-Cov-2 virus in inactivated vs non-inactivated clinical samples of nasopharyngeal swabs in Copan universal transport medium in total 18 samples from infected individuals. FIG. 3A shows the comparison of direct RT-PCR from untreated samples vs RNA extraction followed by RT-PCR. FIG. 3B shows the comparison of direct RT-PCR from untreated vs inactivated for 10 min at 65° C. samples. FIG. 3C shows the comparison of direct RT-PCR from untreated vs inactivated for 10 min at 80° C. samples. The dashed line shows ideal correlation. As shown here, the direct RT-PCR method of the present disclosure provided even lower Ct values than the RNA extraction followed by RT-PCR. Inactivation at 65° C. and 80° C. had no or negligible effect on the outcome, respectively.

FIG. 4 shows the comparison of detection of SARS-Cov-2 virus in 537 samples of nasopharyngeal swabs in transport media by RT-PCR directly from the sample vs conventional RT-PCR after RNA extraction. In the direct RT-PCR assay, 148 samples were positive. In the standard RNA extraction followed by RT-PCR assay, 142 samples were positive. Fourteen samples were positive only in direct RT-PCR, while eight samples were positive only in the standard protocol, but all were extremely weak over 36th cycle. This shows higher sensitivity of the direct detection over the standard RNA isolation and RT-PCR.

FIGS. 5A-5B show the sensitivity of SARS-Cov-2 detection in saliva. FIG. 5A shows comparison of detection of SARS-Cov-2 virus in 445 saliva samples by RT-PCR directly from the sample (x-axis) vs conventional RT-PCR after RNA extraction (y-axis) In the direct RT-PCR assay, 136 samples were positive. In the standard RNA extraction followed by RT-PCR assay, only 108 samples were positive (79%). These results show that the direct RT-PCR from saliva method provided herein is even much more sensitive than standard methods based on RNA extraction. If a sample was detected only in one method, then zero was assigned for the threshold cycle value for the other method and these samples appear on the axis. There were 31 samples positive only in direct RT-PCR appear on x-axis, and 3 samples positive only in RNA extraction followed by RT-PCR appear on y-axis. FIG. 5B shows the comparison of SARS-Cov-2 detection in saliva with the direct RT-PCR vs standard RNA isolation followed by RT-PCR in nasopharyngeal swabs (in total 494 samples each). The samples were different than in FIG. 5A, all were paired samples of saliva and swabs taken at the same time. There were in total 109 positive saliva samples, while only 105 positive samples in swabs. 16 samples were positive only in saliva (including several samples with high viral load) while 12 samples were positive only in swabs, all these samples were very weak (over 35th cycle). This show that direct RT-PCR from saliva is even more sensitive than the standard swab linked with standard RNA isolation and RT-PCR. Detection of SARS-Cov-2 from saliva can thus become the new standard.

It is noted that the drawings are not necessarily to scale. The drawings are intended to depict only typical aspects of the subject matter disclosed herein, and therefore should not be considered as limiting the scope of the disclosure.

### DETAILED DESCRIPTION

Embodiments of the present disclosure generally relate to systems, compositions, kits, and methods for detecting target nucleic acids in a biological sample. In some embodiments, the disclosure provides systems and methods that are ultra-sensitive in detecting target nucleic acids. In other embodiments, the disclosure provides systems and methods that can detect target nucleic acids in various biological samples. In some embodiments, the disclosure provides systems and methods for detecting pathogenic bacterial nucleic acids or human nucleic acids. In some embodiments, the disclosure provides systems and methods that are comparable with methods for detecting viral RNA, such as SARS-Cov-2 RNA from nasopharyngeal swabs in transport medium. In some embodiments, the disclosure provides systems and methods that are compatible with heat-inactivated samples, which would provide safer sample handling. In some embodiments, the disclosure provides systems and methods that are comparable with methods for detecting viral RNA, such as SARS-Cov-2 RNA, from clinical swab samples. In other embodiments, the disclosure provides systems and methods that are advantageous in testing in a pandemic, such as the COVID-19 or influenza pandemic. In some exemplary embodiments, the systems and methods provided herein can detect target nucleic acids in self-collected samples, e.g., saliva samples, which are also much less invasive than the nasopharyngeal swabs. In further embodiments, the systems and methods provided herein are compatible with high throughput automation.

### Definition

The terminology used herein is for the purpose of describing particular cases only and is not intended to be limiting. Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

A “subject” or “individual” refers to a living organism such as a mammal. Examples of subjects include, but are not limited to, minks, horses, cows, camels, sheep, pigs, goats, dogs, cats, rabbits, guinea pigs, rats, mice (e.g., humanized mice), gerbils, non-human primates (e.g., macaques), humans and the like, non-mammals, including, e.g., non-mammalian vertebrates, such as birds (e.g., chickens or ducks) fish (e.g., sharks) or frogs (e.g., Xenopus), and non-mammalian invertebrates, as well as transgenic species thereof. In some embodiments, a subject refers to a single organism. In some embodiments, a subject refers to human. In some embodiments, a subject refers to a patient. A subject from whom samples are obtained can be inflicted with a disease and/or disorder (e.g., one or more allergies, infections, cancers, or autoimmune disorders or the like) and can be compared against a negative control subject which is not affected by the disease. In some embodiments, the subjects can include a group of individuals with or without a disease. In some embodiments, the subject is suspected of having an infectious disease. In some embodiments, the infectious disease is associated with influenza, coronavirus infection, SARS-CoV-1, SARS-CoV-2, MERS, or other respiratory viruses, non-coronavirus, tuberculous or non-tuberculous Mycobacterium. In an exemplary embodiment, the subject is suspected of having SARS-CoV-2.

A “nucleic acid”, refers to either a single nucleotide or at least two nucleotides covalently linked together. “Nucleotide,” “nucleoside,” “nucleotide residue,” and “nucleoside residue,” as used herein, can mean a deoxyribonucleotide or ribonucleotide residue, or other similar nucleoside analogue capable of serving as a component of a primer suitable for use in an amplification reaction (e.g., PCR reaction). Such nucleosides and derivatives thereof can be used as the building blocks of the primers described herein, except where indicated otherwise. Nothing in this application is meant to preclude the utilization of nucleoside derivatives or bases that have been chemical modified to enhance their stability or usefulness in an amplification reaction, provided that the chemical modification does not interfere with their recognition by a polymerase as deoxyguanine, deoxycytidine, deoxythymidine, or deoxyadenine, as appropriate. In some embodiments, nucleotide analogs can stabilize hybrid formation. In some embodiments, nucleotide analogs can destabilize hybrid formation. In some embodiments, nucleotide analogs can enhance hybridization specificity. In some embodiments, nucleotide analogs can reduce hybridization specificity.

In some embodiments, the nucleic acid as used herein comprises polymeric form of nucleotides of any length. In some embodiments, the nucleic acid as used herein can include other molecules, such as another hybridized polynucleotide. In some embodiments, the nucleic acid as used herein include sequences of deoxyribonucleic acid (DNA), ribonucleic acid (RNA), or both. Non-limiting examples of nucleic acids include a gene, a gene fragment, an exon, an intron, intergenic DNA (including, without limitation, heterochromatic DNA), messenger RNA (mRNA), transfer RNA, ribosomal RNA, ribozymes, small interfering RNA (siRNA), cDNA, recombinant polynucleotides, branched polynucleotides, plasmids, vectors, isolated DNA of a sequence, isolated RNA of a sequence, nucleic acid probes, and primers. In some embodiments, the nucleic acids can be isolated from natural sources, recombinant, or artificially synthesized.

In some embodiments, the term “target nucleic acid” refers to a nucleic acid of interest. In some embodiments, the target nucleic acid as used herein can include RNA or DNA. In some embodiments, the target nucleic acid is derived from a pathogen, including algae, bacteria, fungi, viroids, and viruses. In some embodiments, the target nucleic acid can include a viral RNA or viral DNA, bacterial RNA or bacterial DNA, animal RNA or animal DNA, human RNA or human DNA. In some specific embodiments, the target nucleic acid can include viral RNA. In one exemplary embodiment, the target nucleic acid is one or more of SARS-Cov-2 RNA, SARS-1 (2003), MERS, influenza A, influenza B, RSV, other respiratory viruses, Hepatitis A, Hepatitis B, Hepatitis C, or HIV.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

### Systems

In some embodiments, the disclosure provides systems for reverse transcriptase polymerase chain reaction (RT-PCR). The systems can include a buffer, a salt, a mixture of deoxynucleotide triphosphates (dNTPs), a detergent, a reducing agent, an RNA carrier, a DNA polymerase (e.g., a thermostable DNA polymerase), a reverse transcriptase, and an RNAse inhibitor.

As discussed in greater detail below, it has been discovered the addition of one or more RNA carriers is beneficial in an RT-PCR (and in particular, beneficial in an RT-PCR from saliva samples) for improving sensitivity of RNA detection. Conventional systems are not known to include RNA carriers and therefore fail to achieve this benefit. It has also been discovered that relatively high concentrations of the detergent (e.g., a non-ionic detergent having concentration of 0.05%, 0.1%, 0.5% or higher) provide unexpectedly improved results. Contrary to conventional understanding, high concentration of non-ionic detergent does not inhibit the RT-PCR reaction but instead can increase the sensitivity and robustness of the detection.

Depending upon the context, the systems can be either a complete or an incomplete mixture of one or more of the reagents provided herein. The term “buffer” can include its ordinary and customary meaning and further refer to an aqueous solution comprising the various reagents exemplified herein and/or suitable substitutes chosen by a skilled person in the art.

In some embodiments, the buffer can include Tris (tris(hydroxymethyl)aminomethane). In some embodiments, the systems can include Tris at a concentration of about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 41 mM, 42 mM, 43 mM, 44 mM, 45 mM, 46 mM, 47 mM, 48 mM, 49 mM, 50 mM, 55 mM, 60 mM, 65 mM, 70 mM, 75 mM, 80 mM, 85 mM, 90 mM, 95 mM, or 100 mM. In some embodiments, the systems can include Tris at a concentration within the range from about 1 mM to about 500 mM, about 5 to about 250 mM, about 7 to about 150 mM, about 10 to about 100 mM, about 20 to about 80 mM, about 30 to about 70 mM, or about 40 to about 60 mM. In an exemplary embodiment, the systems comprise Tris at a concentration in the range from about 10 to about 40 mM.

In other embodiments, the buffer can include at least one of Bis-tris-propane, PIPES (piperazine-N,N′-bis(2-ethanesulfonic acid), MOPS (3-(N-morpholino) propanesulfonic acid), or HEPES (4-(2-hydroxyethyl)-1-piperazineethanesulfonic acid). In some embodiments, the buffer can further include at least one of glycerol, dimethyl sulfoxide (DMSO), Bovine serum albumin (BSA), or casein. In some embodiments, glycerol or DMSO alters annealing of the primers. In other embodiments, BSA or casein improves the stability. It is noted that other reagents known for use in any PCR or RT-PCR are also encompassed by the present disclosure. One skilled in the art would understand how to choose the optimal buffer systems.

In some embodiments, the salt includes one or more salts. The one or more salts can include, but are not limited to, at least one of potassium chloride, ammonium sulfate, magnesium chloride or magnesium sulfate

In an embodiment, the salt can be potassium chloride. In some embodiments, the concentration of potassium chloride is about 5 to about 200 mM, about 10 to about 150 mM, about 20 to about 125 mM, about 30 to about 120 mM, about 40 to about 110 mM, about 50 to about 100 mM, about 60 to about 90 mM, about 70 to about 80 mM. In an exemplary embodiment, the concentration of potassium chloride is about 50 to about 100 mM. In other embodiments, the concentration of potassium chloride is about 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the salt can include magnesium chloride. In some embodiments, the concentration of magnesium chloride is about 0.5 mM to about 10 mM, about 1 mM to about 8 mM, or about 1 mM to about 5 mM. In an exemplary embodiment, the concentration of magnesium chloride is about 2 mM to about 5 mM. In other embodiments, the concentration of magnesium chloride is about 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, or 10 mM.

In some embodiments, the salt comprises ammonium sulfate. In some embodiments, the concentration of ammonium sulfate is about 5 to about 200 mM, about 10 to about 100 mM, about 20 to about 50 mM. In an exemplary embodiment, the concentration of ammonium sulfate is about 20 to about 50 mM. In other embodiments, the concentration of ammonium sulfate is about 20 mM, 25 mM, 30 mM, 35 mM, 40 mM, 45 mM, 50 mM.

In some embodiments, the salt comprises magnesium sulfate. In some embodiments, the concentration of magnesium sulfate is about 0.5 mM to about 10 mM, about 1 mM to about 8 mM, or about 1 mM to about 5 mM. In an exemplary embodiment, the concentration of magnesium sulfate is about 2 mM to about 5 mM. In other embodiments, the concentration of magnesium sulfate is about 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, or 10 mM

In some embodiments, the buffer comprises a mixture of dNTPs. The mixture of dNTPs can include one or more of dATP, dCTP, dTTP, dUTP, or dGTP. In some embodiments, the buffer is a mixture of dNTPs, including dATP, dCTP, dTTP, and dGTP. In other embodiments, the buffer is a mixture of dNTPs including dATP, dCTP, dUTP, and dGTP. Generally, the concentration of dNTPs in a PCR is within the range of about 10 μM to about 1 mM, about 20 μM to about 900 μM, about 30 μM to about 800 μM, about 40 μM to about 700 μM, about 50 μM to about 600 μM, about 60 μM to about 500 μM, about 7011M to about 400 μM, about 80 μM to about 300 μM, about 90 μM to about 200 μM, or about 100 μM to about 150 μM each dNTP. In some embodiments, each of the dATP, dCTP, dTTP, and dGTP has a final concentration in the systems of about 0.05 to about 0.5 mM each. In some embodiments, each of the dATP, dCTP, dTTP, and dGTP has a final concentration in the systems within the range from about 0.1 to about 0.4 mM.

In some embodiments, the DNA polymerase is a thermostable DNA polymerase. Thermostable DNA polymerase is able to catalyze the extension reaction on the DNA template. In some embodiments, the thermostable DNA polymerase comprises at least one of a Taq polymerase, a Tth Polymerase, a Bst polymerase, or a Z05 polymerase. In some embodiments, the thermostable DNA polymerase is a wild-type enzyme. In other embodiments, the thermostable DNA polymerase can include one or more single point mutations. In some embodiments, the thermostable DNA polymerase is on N-terminus, C-terminus, internally truncated, or fused to another peptide or protein. In some embodiments, the thermostable DNA polymerase does not have the 5′-3′ exonuclease activity. In other embodiments, the thermostable DNA polymerase has the 5′-3′ exonuclease activity and is referred to as exo+Taq DNA polymerase.

In some embodiments, the systems provided herein can include a thermostable DNA polymerase with a concentration higher than about 0.01 U/μL. In some embodiments, the concentration of the thermostable DNA polymerase is higher than about 0.02 U/μL. In some embodiments, the concentration of the thermostable DNA polymerase is higher than about U/μL. In some embodiments, the concentration of the thermostable DNA polymerase is higher than about 0.04 U/μL. In some embodiments, the concentration of the thermostable DNA polymerase is higher than about 0.05 U/μL. In some embodiments, the concentration of the thermostable DNA polymerase is higher than about 0.06 U/μL. In some embodiments, the concentration of the thermostable DNA polymerase is higher than about 0.07 U/μL. In some embodiments, the concentration of the thermostable DNA polymerase is higher than about U/μL. In some embodiments, the concentration of the thermostable DNA polymerase is higher than about 0.09 U/μL. In some embodiments, the concentration of the thermostable DNA polymerase is higher than about 1 U/μL. In some embodiments, the concentration of the thermostable DNA polymerase is higher than about 1.5 U/μL. In some embodiments, the concentration of the thermostable DNA polymerase is higher than about 2 U/μL. In some embodiments, the concentration of the thermostable DNA polymerase is higher than about 2.5 U/μL.

In some embodiments, the concentration of the thermostable DNA polymerase is within the range from about 0.01 to about 2.5 U/μL. In some embodiments, the concentration of the thermostable DNA polymerase is in the range from about 0.02 to about 2 U/μL. In some embodiments, the concentration of the thermostable DNA polymerase is within the range from about 0.03 to about 1.8 U/μL. In some embodiments, the concentration of the thermostable DNA polymerase is within the range from about 0.04 to about 1.6 U/μL. In some embodiments, the concentration of the thermostable DNA polymerase is within the range from about 0.05 to about 1.4 U/μL. In some embodiments, the concentration of the thermostable DNA polymerase is within the range from about 0.06 to about 1.2 U/μL. In some embodiments, the concentration of the thermostable DNA polymerase is within the range from about 0.07 to about 1.0 U/μL. In some embodiments, the concentration of the thermostable DNA polymerase is within the range from about 0.08 to about 0.9 U/μL. In some embodiments, the concentration of the thermostable DNA polymerase is within the range from about 0.09 to about 0.8 U/μL. In some embodiments, the concentration of the thermostable DNA polymerase is within the range from about 0.1 to about 0.7 U/μL. In some embodiments, the concentration of the thermostable DNA polymerase is within the range from about 0.2 to about 0.6 U/μL. In some embodiments, the concentration of the thermostable DNA polymerase is within the range from about 0.3 to about 0.5 U/μL. In some specific embodiments, the concentration of the thermostable DNA polymerase is within the range from about 0.05 to about 0.2 U/μL.

As molecular biology and diagnostic applications have become more demanding and sensitive, the ability to control enzymatic activities, and therefore reaction specificity, has naturally followed. Thus, in some embodiments, the Taq DNA polymerase used herein involves an activity control mechanism. For example, a modifier such as an antibody, affibody, aptamer, or other chemical modification can be added to inhibit PCR enzyme activity at room temperature. The modifier is then removed during the initial heating step of PCR resulting in a functional Taq DNA polymerase. However, it is understood that other Taq DNA polymerase activity control mechanisms are also contemplated by the present disclosure.

In some embodiments, the Taq DNA polymerase is inactivated by aptamer-oligonucleotides. The aptamer-based hot start approach involves the selection of specific, modified aptamers for control of any enzymatic target of interest. In some embodiments, the Taq DNA polymerase is inactivated by aptamer-oligonucleotides with a concentration of about 1 nM to about 500 nM at about room temperature (e.g., about 20° C. to about 22° C.; about 68° F. to about 72° F.) or at elevated temperatures up to about 55° C. In some embodiments, the Taq DNA polymerase is inactivated by aptamer-oligonucleotides with a concentration of about 2 nM to about 450 nM at about room temperature. In some embodiments, the Taq DNA polymerase is inactivated by aptamer-oligonucleotides with a concentration of about 3 nM to about 400 nM at about room temperature. In some embodiments, the Taq DNA polymerase is inactivated by aptamer-oligonucleotides with a concentration of about 4 nM to about 350 nM at about room temperature. In some embodiments, the Taq DNA polymerase is inactivated by aptamer-oligonucleotides with a concentration of about 5 nM to about 300 nM at about room temperature. In some embodiments, the Taq DNA polymerase is inactivated by aptamer-oligonucleotides with a concentration of about 6 nM to about 275 nM at about room temperature. In some embodiments, the Taq DNA polymerase is inactivated by aptamer-oligonucleotides with a concentration of about 7 nM to about 250 nM at about room temperature. In some embodiments, the Taq DNA polymerase is inactivated by aptamer-oligonucleotides with a concentration of about 8 nM to about 225 nM at about room temperature. In some embodiments, the Taq DNA polymerase is inactivated by aptamer-oligonucleotides with a concentration of about 9 nM to about 210 nM at about room temperature. In some embodiments, the Taq DNA polymerase is inactivated by aptamer-oligonucleotides with a concentration of about 10 nM to about 200 nM at about room temperature. The aptamer-oligonucleotides are designed to inhibit the Taq DNA polymerase at temperatures below but not at or above 60° C.

In some embodiments, the enzymatic activity of the Taq DNA polymerase is controlled (e.g., reversibly inactivated) by bound anti-DNA polymerase antibodies. An anti-DNA polymerase antibody is a monoclonal antibody that binds Taq polymerase and inhibits its activity until reaction temperature is elevated. At that point, the anti-DNA polymerase Antibody is denatured and releases its hold on Taq polymerase, allowing DNA synthesis to proceed. Such antibodies are commercially available and are encompassed by the present disclosure. Exemplary anti-DNA polymerase Antibodies include, without being limited to AmpliTaq™ from Applied Biosystems, Platinum II Taq™ from Invitrogen, JumpStart™ from Sigma Aldrich, and Platinum® Taq from Thermo Fisher Scientific.

In other embodiments, the enzymatic activity of the Taq DNA polymerase is controlled (e.g., reversibly inactivated) by chemical modifications. Chemical modifications can include modification with a dicarboxylic acid anhydride, citraconic anhydride, cis-aconitic anhydride, maleic anhydride, changing at least one Lysine residue to an Arginine residue, or fusion with a protein such as Sso7d protein. Such chemically modified Taq DNA polymerases are commercially available and are encompassed by the present disclosure. Exemplary chemically modified Taq DNA polymerases include DreamTaq™ from Thermo Fisher Scientific, AmpliTaq™ from Roche, FlashTaq™ from Empirical Bioscience, and TaqTivate™ from Molecular Innovations.

In the above embodiments, the DNA polymerase is also referred to as a hot start DNA polymerase. In some embodiments, the hot start DNA polymerase is a hot start Taq DNA polymerase. In some embodiments, the hot start Taq DNA polymerase does not have the 5′-3′ exonuclease activity. In some embodiments, the hot start Taq DNA polymerase has the 5′-3′ exonuclease activity.

In some embodiments, the systems further comprise a reverse transcriptase. Reverse transcriptases generally refer to RNA-directed DNA polymerases that were first identified as part of the retroviral life cycle. Typically, the reverse transcriptase is active at about 37 to about 42° C. In some embodiments, the reverse transcriptase is a thermostable reverse transcriptase. Thermostable reverse transcriptases generally refer to the reverse transcriptases that retain part or all of their catalytic activities under elevated temperatures. Typically, a thermostable reverse transcriptase is active at or above 50° C.

In some embodiments, the reverse transcriptase is a wild-type enzyme. In some embodiments, the reverse transcriptase comprises one or more single point mutations. In other embodiments, the reverse transcriptase is truncated. In some embodiments, the reverse transcriptase is an RNAse H⁻ mutant. In other embodiments, the reverse transcriptase is on N-terminus, C-terminus, internally truncated, or fused to another peptide or protein.

Exemplary reverse transcriptase includes, without limitations, M-MLV reverse transcriptase, AMV reverse transcriptase, FeLV reverse transcriptase, ExcellScript Thermostable M-MuLV Reverse Transcriptase, RapiDxFire™ Thermostable Reverse Transcriptase, RocketScript™ Thermostable Reverse Transcriptase, Superscript II Reverse Transcriptase, Superscript III Reverse Transcriptase, Superscript IV Reverse Transcriptase, Protoscript® II Reverse Transcriptase, or Maxima H minus Reverse Transcriptase. Different reverse transcriptases have different characteristics, and some are better suited to specific applications than others. The downstream application, the length of the target RNA, presence of complex RNA secondary structure and an enzyme's level of RNase H activity are all considerations when choosing the right reverse transcriptase. One skilled in the art would understand how to choose a suitable reverse transcriptase for a specific application.

In some embodiments, the concentration of the reverse transcriptase is higher than about 0.01 U/μL. In some embodiments, the concentration of the reverse transcriptase is higher than about 0.02 U/μL. In some embodiments, the concentration of the reverse transcriptase is higher than about 0.03 U/μL. In some embodiments, the concentration of the reverse transcriptase is higher than about 0.04 U/μL. In some embodiments, the concentration of the reverse transcriptase is higher than about 0.05 U/μL. In some embodiments, the concentration of the reverse transcriptase is higher than about 0.06 U/μL. In some embodiments, the concentration of the reverse transcriptase is higher than about 0.07 U/μL. In some embodiments, the concentration of the reverse transcriptase is higher than about 0.08 U/μL. In some embodiments, the concentration of the reverse transcriptase is higher than about 0.09 U/μL. In some embodiments, the concentration of the reverse transcriptase is higher than about 0.1 U/μL. In some embodiments, the concentration of the reverse transcriptase is higher than about 0.2 U/μL. In some embodiments, the concentration of the reverse transcriptase is higher than about 0.3 U/μL. In some embodiments, the concentration of the reverse transcriptase is higher than about 0.4 U/μL. In some embodiments, the concentration of the reverse transcriptase is higher than about 0.5 U/μL. In some embodiments, the concentration of the reverse transcriptase is within the range from about 0.01 to about 1 U/μL, about 0.02 to about 0.9 U/μL, about 0.03 to about 0.8 U/μL, about 0.04 to about 0.7 U/μL, about 0.05 to about 0.6 U/μL, or about 0.05 to about 0.5 U/μL. In one exemplary embodiment, the concentration of the reverse transcriptase is in the range from about 0.05 to about 0.5 U/μL.

In some embodiments, the reverse transcriptase is inactivated by aptamer-oligonucleotides at about room temperature (e.g., about 20° C. to about 22° C.; about 68° F. to about 72° F.) or at elevated temperatures up to about 30° C. or even up to about 45° C. In such embodiments, the reverse transcriptase is referred to as warm-started. The aptamer-based warm-start approach involves the selection of specific, modified aptamers for control of any enzymatic target of interest. In some embodiments, the reverse transcriptase is inactivated by aptamer-oligonucleotides with a concentration of about 1 nM to about 500 nM at about room temperature. In some embodiments, the reverse transcriptase is inactivated by aptamer-oligonucleotides with a concentration of about 2 nM to about 450 nM at about room temperature. In some embodiments, the reverse transcriptase is inactivated by aptamer-oligonucleotides with a concentration of about 3 nM to about 400 nM at about room temperature. In some embodiments, the reverse transcriptase is inactivated by aptamer-oligonucleotides with a concentration of about 4 nM to about 350 nM at about room temperature. In some embodiments, the reverse transcriptase is inactivated by aptamer-oligonucleotides with a concentration of about 5 nM to about 300 nM at about room temperature. In some embodiments, the reverse transcriptase is inactivated by aptamer-oligonucleotides with a concentration of about 6 nM to about 275 nM at about room temperature. In some embodiments, the reverse transcriptase is inactivated by aptamer-oligonucleotides with a concentration of about 7 nM to about 250 nM at about room temperature. In some embodiments, the reverse transcriptase is inactivated by aptamer-oligonucleotides with a concentration of about 8 nM to about 225 nM at about room temperature. In some embodiments, the reverse transcriptase is inactivated by aptamer-oligonucleotides with a concentration of about 9 nM to about 210 nM at about room temperature. In some embodiments, the reverse transcriptase is inactivated by aptamer-oligonucleotides with a concentration of about 10 nM to about 200 nM at about room temperature. In some embodiments, the aptamer-oligonucleotides are designed to inhibit the reverse transcriptase at temperatures below about 37° C. for non-thermal stable reverse transcriptases and up to 50° C. but not above 50° C. for thermostable reverse transcriptases.

In some embodiments, the systems can further include one or more reducing agents. As used herein, the term “reducing agent” can adopt its ordinary and customary meaning and can further include any element, compound, or combination of elements and/or compounds that reduces or breaks a disulfide bond. Reducing agents are generally known and used in the art. Exemplary reducing agents include, without limitations, Dithiothreitol (DTT), β-mercaptoethanol, tris(2-carboxyethyl)phosphine (TCEP), glutathione, acetyl L-cystein, acetyl D-cystein, L-Cysteine methyl ester, D-Cysteine methyl ester, L-Cysteine methyl ester, D-Cysteine methyl ester, N-Formyl-L-cysteine, Tris(hydroxypropyl)phosphine, Tris(hydroxymethyl)phosphine, Sodium triacetoxyborohydride, 1,2-Ethanedithiol, 2-Mercaptopropan-1-ol, 3-Mercaptopropan-1-ol, 1-mercaptopropan-2-ol, Thioglycolic acid and a salt, Dithiothreitol, 2-Mercaptobenzoic acid, 3-Mercaptobenzoic acid, 4-Mercaptobenzoic acid, 4-Mercaptobutan-1-ol, Cysteamine, homocysteine, N-Acetyl-L-homocysteine, L-homocysteine methyl ester, 3-mercaptobutanol, Dihydrolipoic acid, dithiobutylamine, sodium sulfite, NADH, FADH₂, 2,3-Pyrazinedithiol, thiourea, or thiolactic acid.

In some embodiments, the reducing agent is DTT. In some embodiments, the concentration of DTT is higher than about 0.001 mM. In some embodiments, the concentration of DTT is higher than about 0.002 mM. In some embodiments, the concentration of DTT is higher than about 0.003 mM. In some embodiments, the concentration of DTT is higher than about 0.004 mM. In some embodiments, the concentration of DTT is higher than about 0.005 mM. In some embodiments, the concentration of DTT is higher than about 0.006 mM. In some embodiments, the concentration of DTT is higher than about 0.007 mM. In some embodiments, the concentration of DTT is higher than about 0.008 mM. In some embodiments, the concentration of DTT is higher than about 0.009 mM. In some embodiments, the concentration of DTT is higher than about 0.01 mM. In some embodiments, the concentration of DTT is within the range from about 0.05 to about 1.5 mM. In some embodiments, the concentration of DTT is within the range from about 0.06 to about 1.4 mM. In some embodiments, the concentration of DTT is in the range from about 0.07 to about 1.3 mM. In some embodiments, the concentration of DTT is within the range from about 0.08 to about 1.2 mM. In some embodiments, the concentration of DTT is within the range from about 0.09 to about 1.1 mM. In one exemplary embodiment, the concentration of DTT is within the range from about 0.1 to about 1.0 mM.

As noted above, embodiments of the systems can include one or more RNAse inhibitors. Various RNase inhibitors can generally refer to compounds intended to inactivate ribonuclease enzymes, which degrade RNA. In some embodiments, the RNAse inhibitor remains active at elevated temperatures of up to about 40° C., up to about 50° C. or even up to about 60° C.

Because RNases fulfill a broad range of biological roles, they are among the most common enzymes. Even traces of RNase can nick the RNA, causing shortened cDNA products, low yields, and reduced RT-PCR sensitivity. RNase inhibitors are commonly used as a precautionary measure in enzymatic manipulations of RNA, such as in RT-PCRs, to inhibit and control for RNases contaminants.

Exemplary the RNAse inhibitors can include, without limitations, porcine liver RNAse inhibitors, human placental RNAse inhibitors, murine RNAse inhibitors, rat lung RNAse inhibitors, or rat liver RNAse inhibitors. Exemplary RNAse inhibitors include, without limitations, RNasin, RNAsin Plus, Ribolock, RNAseOUT™, or Superase In™.

In some embodiments, the concentration of the RNAse inhibitor is within the range of about 0.01 U/μL to about 0.1 U/μL. In some embodiments, the concentration of RNAse inhibitor is about 0.01 U/μL, 0.02 U/μL, 0.03 U/μL, 0.04 U/μL, or 0.05 U/μL, 0.06 U/μL, 0.07 U/μL, 0.08 U/μL, 0.09 U/μL or 0.1 U/μL. In other embodiments, the concentration of the RNAse inhibitor is within the range of about 0.05 U/μL, to about 0.5 U/μL. In some embodiments, the concentration of the RNAse inhibitor is within the range of about 0.06 U/μL to about 0.4 U/μL. In other embodiments, the concentration of the RNAse inhibitor is within the range of about 0.07 U/μL to about 0.3 U/μL. In other embodiments, the concentration of the RNAse inhibitor is within the range of about 0.08 U/μL to about 0.25 U/μL. In some embodiments, the concentration of RNAse inhibitor is higher than about 0.1 U/μL. In other embodiments, the concentration of RNAse inhibitor is higher than about 0.2 U/μL. In other embodiments, the concentration of RNAse inhibitor is higher than about 0.3 U/μL. In other embodiments, the concentration of RNAse inhibitor is higher than about 0.4 U/μL. In other embodiments, the concentration of RNAse inhibitor is higher than about 0.5 U/μL.

In some embodiments, the RNAse inhibitor can include one or more single point mutations. In other embodiments, the RNAse inhibitor is on N-terminus, C-terminus, internally truncated, or fused to another peptide or protein.

In some embodiments, the systems provided herein can include both a reverse transcriptase and an RNAse inhibitor as described herein. In some embodiments, the reverse transcriptase and the RNAse inhibitor are added to the systems simultaneously.

Existing RT-PCR mixes do not contain RNA carriers. However, in some embodiments, the systems provided herein can include one or more RNA carriers. It is been discovered when testing addition of RNA carriers on multiple sample types (e.g., blood serum, blood plasma, saliva, nasal swab, nasopharyngeal swab, nasal wash, mouth swab, mouth wash, seminal plasma, or, etc.) from multiple donors that the addition of RNA carries has unexpected benefits. Notably, if the RNA is detected directly in the sample without prior RNA extraction (e.g., the RNA is not purified from the sample, the sample is added directly to the RT/PCR reaction), addition of one or more RNA carriers can improve the sensitivity of RNA detection. In particular, significant improvement has been observed in saliva samples, where approximately 100% recovery of the target RNA was achieved. Furthermore, the limit of detection of the target RNA was in the range of 1 to 5 copies. Corresponding control tests performed without addition of one or more RNA carriers were not able to achieve this detection limit. Thus, in some instances, the addition of one or more RNA carriers is beneficial in an RT-PCR, and in particular, beneficial in an RT-PCR from saliva samples.

Examples of RNA carriers can include, but are not limited to, polynucleotides such as DNA and/or RNA, or polypeptides. Examples of DNA carriers can also include plasmids, vectors, polyadenylated DNA, and DNA polynucleotides. Examples of RNA carriers can further include, but are not limited to, polyadenylated RNA, phage RNA, phage MS2 RNA, E. coli RNA, yeast RNA, yeast tRNA, mammalian RNA, mammalian tRNA, short polyadenylated synthetic ribonucleotides and RNA polynucleotides. The RNA carrier may be a polyadenylated RNA. Alternatively, the RNA carrier may be a non-polyadenylated RNA. In some embodiments, the carrier is from a bacteria, yeast, or virus. For example, the carrier may be a polynucleotide or a polypeptide derived from a bacteria, yeast or virus. For example, the carrier is a protein from Bacillus subtilis. In another example, the carrier is a polynucleotide from Escherichia coli (E. coli). Alternatively, the carrier is a polynucleotide or peptide from a mammal (e.g., human, mouse, goat, rat, cow, sheep, pig, dog, or rabbit), avian, amphibian, or reptile. In some embodiments, the RNA carrier comprises a polyinosinic acid, a polyinosinic-polycytidylic acid, or a polyadenosine. In a specific embodiment, the RNA carrier comprises a polyinosinic acid. In another specific embodiment, the RNA carrier comprises a polyadenosine. In a specific embodiment, the RNA carrier comprises a polyadeylic acid. In some embodiments, the carrier improves the effect of the RNAse inhibitors, when added simultaneously. Thus, in some embodiments, the RNA carrier is added to systems simultaneously with the reverse transcriptase and the RNAse inhibitor.

In some embodiments, the systems provided herein can include RNA carriers with a concentration in the range from about 0.0005 to about 0.05 mg/mL. In some embodiments, the concentration of RNA carriers is within the range from about 0.00004 to about 0.15 mg/mL. In some embodiments, the concentration of RNA carriers is within the range from about 0.00005 to about 0.14 mg/mL. In some embodiments, the concentration of RNA carriers is within the range from about 0.00006 to about 0.13 mg/mL. In some embodiments, the concentration of RNA carriers is within the range from about 0.00007 to about 0.12 mg/mL. In some embodiments, the concentration of RNA carriers is within the range from about 0.00008 to about 0.11 mg/mL. In some embodiments, the concentration of RNA carriers is within the range from about 0.00009 to about 0.10 mg/mL. In some embodiments, the concentration of RNA carriers is within the range from about 0.0001 to about 0.09 mg/mL. In some embodiments, the concentration of RNA carriers is within the range from about 0.0002 to about 0.08 mg/mL. In some embodiments, the concentration of RNA carriers is within the range from about 0.0003 to about 0.07 mg/mL. In some embodiments, the concentration of RNA carriers is within the range from about 0.0004 to about 0.06 mg/mL. In some embodiments, the concentration of RNA carriers is within the range from about 0.0005 to about 0.10 mg/mL. In some embodiments, the concentration of RNA carriers is in the range from about 0.0006 to about 0.09 mg/mL. In some embodiments, the concentration of RNA carriers is within the range from about 0.0007 to about 0.08 mg/mL. In some embodiments, the concentration of RNA carriers is within the range from about 0.0008 to about 0.07 mg/mL. In some embodiments, the concentration of RNA carriers is within the range from about 0.0009 to about 0.06 mg/mL. In some embodiments, the concentration of RNA carriers is within the range from about 0.001 to about 0.05 mg/mL. In some embodiments, the concentration of RNA carriers is within the range from about 0.002 to about 0.04 mg/mL. In some embodiments, the concentration of RNA carriers is within the range from about 0.003 to about 0.03 mg/mL. In some embodiments, the concentration of RNA carriers is within the range from about 0.004 to about 0.02 mg/mL. In some embodiments, the systems provided herein can include RNA carriers with a concentration within the range from about 0.002 to about 0.01 mg/mL.

In certain exemplary embodiments, the RNA carrier is polyinosinic acid and the concentration of polyinosinic acid is within the range from about 0.0005 to about 0.05 mg/mL, inclusive. In other exemplary embodiments, the RNA carrier is polyinosinic acid and the concentration of polyinosinic acid is within the range from about 0.002 to about 0.01 mg/mL, inclusive.

PCR is usually carried out in a buffer that provides a suitable chemical environment for activities of the enzymes, such as DNA polymerase. The buffer pH is usually 8.8 or more and is often stabilized by Tris, Tris-HCl, and the like. However, it has been discovered that lowering the pH of the systems provided herein can improve the robustness of the detection of the target nucleic acid. Without being bound by theory, it is believed that the pH used in most presently available RT-PCR buffers (8.8 or more at 25° C.) is not well suited and leads to failure of detection of target nucleic acids, such as viral RNAs, in some samples.

In some embodiments, the systems provided herein have a pH lower than 8.8 at about 25° C. In some embodiments, the systems provided herein have a pH in the range of about 7.0 to less than or equal to about 8.8, about 8.0 to about 8.8, about 8.2 to about 8.8, about 8.1 to about 8.7, or about 8.2 to about 8.6. In some embodiments, the systems provided herein have a pH in the range of about 8.4 to about 8.6. In some embodiments, the systems provided herein have a pH of about 8.4, about 8.5, about 8.6, or any pH therebetween.

As indicated above, embodiments of the systems provided herein can further include a detergent, such as a nonionic detergent. It has been discovered that relatively high concentrations of non-ionic detergent (e.g., 0.05%, 0.1%, 0.5% or higher) provide unexpected results. In one aspect, high concentration of non-ionic detergent does not inhibit the RT-PCR reaction. On the contrary, higher efficiency of the RT-PCR detection from actual samples containing intact viruses has been observed. In another aspect, the high concentration of these detergents helped to overcome inhibition of the RT-PCR reaction by inhibitors present in some of the biological samples. Thus, high concentrations of the nonionic detergents can increase the sensitivity and robustness of the detection.

In some embodiments, the non-ionic detergent can include at least one non-ionic detergent. The at least one non-ionic detergent can include Tween 20 (Polyoxyethylene (20) sorbitan monolaurate), Tween 40 (polyoxyethylene sorbitan monopalmitate), Tween 80 (Polyoxyethylene (20) sorbitan monooleate), Nonidet™ P-40 (octylphenoxypolyethoxyethanol), NP-40 (nonylphenoxypolyethoxyethanol), or Triton™ X-100 (2-[4-(2,4,4-trimethylpentan-2-yl)phenoxy]ethanol), C₁₂E₈ (Dodecyl octaethylene glycol ether) or dodecylmaltoside (DDM). It can be appreciated that this list of non-ionic detergents is not exhaustive and that other non-ionic detergents can be employed without limit.

In some embodiments, the systems provided herein can include up to 1% or even 2% of nonionic detergent without any loss of signal. In some embodiments, incorporating a nonionic detergent at final concentration of about 0.1% to about 1.0% leads to increased sensitivity in the detection of the target nucleic acids in a sample, such as intact viruses in clinical samples or in samples spiked with viral culture.

In some embodiments, the concentration of the nonionic detergent in the systems is within the range from about 0.05% to about 5%. In some embodiments, the concentration of the nonionic detergent in the systems is within the range from about 0.01% to about 5.5%. In some embodiments, the concentration of the nonionic detergent in the systems is within the range from about 0.02% to about 5.0%. In some embodiments, the concentration of the nonionic detergent in the systems is within the range from about 0.03% to about 4.5%. In some embodiments, the concentration of the nonionic detergent within the systems is in the range from about 0.04% to about 4.0%. In some embodiments, the concentration of the nonionic detergent in the systems is within the range from about 0.05% to about 3.5%. In some embodiments, the concentration of the nonionic detergent in the systems is within the range from about 0.06% to about 3.0%. In some embodiments, the concentration of the nonionic detergent in the systems is within the range from about 0.07% to about 2.5%. In some embodiments, the concentration of the nonionic detergent in the systems is within the range from about 0.08% to about 2.0%. In some embodiments, the concentration of the nonionic detergent in the systems is within the range from about 0.09% to about 1.5%. In one exemplary embodiment, the concentration of the nonionic detergent in the systems is within the range from about 0.1% to about 1.0%. In other exemplary embodiment, the concentration of the nonionic detergent in the systems is within the range from about 0.2% to about 1.0%. In one exemplary embodiment, the concentration of the nonionic detergent in the systems is about 0.5%.

In some embodiments, the systems provided herein can include all of the forgoing reagents. In some embodiments, combination of all the forgoing reagents leads to a system that can detect a single copy of a target nucleic acid, such as SARS-Cov-2 RNA, in multiple biological samples.

As discussed above, embodiments of the systems provided herein include compositions for RT-PCR. In certain embodiments, the systems can include a RT-PCR mixture. It is understood that any one or more of the reagents provided herein can be in the same container or in separate containers. In some embodiments, the system is provided as a concentrate of a mixture of any one or more of the reagents provided herein.

In further embodiments, the systems provided herein can include one or more primers. In some embodiments, the one or more primers are configured to hybridize to the target nucleic acids. Typically, forward and reverse primers are designed to anneal to the target nucleic acid sequences (e.g., the target DNA or RNA) during the PCR annealing step and then extended by the polymerase. In one step RT-PCR, at least one of the primers anneals to the target RNA and is then elongated by the reverse transcriptase in a process called reverse transcription of RNA to DNA.

In some embodiments, the systems provided herein can include a pair of forward and reverse primers and optionally a probe for each target. In some embodiments, the probe is dual-labeled, e.g., it can contain fluorophore at its 5′ termini and a non-fluorescent quencher on its 3′ termini. An example of dual-labeled probes is the Taqman™ probes. In some embodiments, the systems provided herein do not include a probe. In such embodiments, nonspecific staining of double stranded DNA can be used (e.g., SYBR Green I dye). In some embodiments, the probes can be used for selective detection of multiple targets (e.g., of multiple gene targets from one virus, or of gene targets from multiple viruses) in one reaction.

In some embodiments, the target nucleic acid is derived from a biological sample. As used herein, the term “sample” or “biological sample” can adopt its ordinary and customary meaning and can also general refers to any material that is taken from its native or natural state, to facilitate any desirable manipulation, further processing, and/or modification. In some embodiments, the sample refers to a biological material that is taken from a subject.

In some embodiments, the biological sample includes at least one of blood, blood serum, blood plasma (anticoagulated with EDTA or heparin or citrate), saliva, nasal swab, mouth swab, nasopharyngeal swab, nasal wash, mouth wash, seminal plasma, or urine, or any combination thereof. In other embodiments, the biological sample can also include peripheral blood mononuclear cells (PBMCs), cells, tissues, biopsies, cerebrospinal fluid, bile, lymph fluid, and stool. In other embodiments, the biological sample can also include blood plasma with EDTA, heparin, or citrate, all at least 10% of the final reaction volume. In some embodiments, the sample can include non-treated saliva, heat inactivated saliva, proteinase K treated and heat inactivated saliva (all up to 25% of the final reaction volume), or various viral transport media. One example of viral transport media is Copan Universal Transport Media (UTM™). Another example of viral transport media is phosphate buffered saline (PBS). In some embodiments, the sample can comprise up to 25% of the final reaction volume of transport media or PBS.

A sample can be further isolated and/or purified from its native or natural state. Alternatively, a sample can be derived from cell or tissue cultures in vitro. In some embodiments, a sample can be processed to extract a protein (e.g., antibody, enzyme, soluble protein, insoluble protein) or nucleic acids (e.g., RNA, DNA). In some embodiments, no prior sample treatment was necessary. However, embodiments of the systems provided herein are compatible with pretreated samples, such as dilution, centrifugation, heating, addition of protease K, addition of various viral transport media, or addition of specialty chemicals.

In some embodiments, embodiments of the systems of the present disclosure further comprise one or more probes. In some embodiments, the probes can include dual labeled probes. In some embodiments, the dual labeled probes target sequences in the PCR amplicons (hybridizing to the target sequence between the forward and reverse primer), which are cleaved by the 5′-3′ exo+DNA polymerase during PCR. In some embodiments, these probes can be labeled with different fluorophores, and therefore, the target nucleic acids can be detected in different dye channels. In some embodiments, the probes can be configured to target regions in one or more of a SARS-CoV-2 EndoRNAse gene, a SARS-CoV-2 Spike gene, a SARS-CoV-2 ORF1ab gene, a SARS-CoV-2 RdRPgene, a SARS-CoV-2 E gene, or a SARS-CoV-2 N gene. In some embodiments, the systems of the present disclosure comprise primer pair and dual labeled probe configured to target a region in a SARS-CoV-2 EndoRNAse gene. In some embodiments, the systems of the present disclosure comprise primer pair and dual labeled probe configured to targeting a region in a viral Spike gene. In some embodiments, the systems of the present disclosure comprise a primer pair and dual labeled probes configured to target a region in a SARS-CoV-2 EndoRNAse gene and a SARS-CoV-2 Spike gene, respectively.

In some embodiments, the systems of the present disclosure comprise one or more primers and/or one or more probes targeting one or more regions in the human internal control gene(s). In some embodiments, the primers and/or the probes are designed to target a region in the human RNAse P gene. However, one skilled in the art would know how to design select alternative internal control targets.

In some embodiments, the systems of the present disclosure comprise an external RNA and/or an external DNA control. In some embodiments, the RNA and/or the DNA control is added externally to the sample prior to analysis; a primer pair and a probe targeting this external control is used for its detection. In some embodiments, the external control is naturally occurring RNA or DNA, synthetic RNA or DNA, or encapsulated RNA or DNA.

In some embodiments, the probes are fluorescently labeled. Many fluorescent PCR primer- and probe-based chemistries have been devised and are available from different commercial vendors, including, without limitations, Hydrolysis (TaqMan™) probes, Molecular beacons, Dual hybridization probes, Eclipse probes, Amplifluor® assays, Scorpions PCR primers, LUX PCR primers, and QZyme PCR primers. Common fluorescent labels include, without limitations, FAM, HEX, Cy5, Cy5.5, Cy3, Cy3.5, NED, ROX, TAMRA, TET, VIC, JOE, Cal Fluor Orange, Cal Fluor Gold, Cal Fluor Red 590, Cal Fluor Red 610, Cal Fluor Red 635, Pulsar 650, Quasar 570, Quasar 670, Quasar 705, and Texas Red.

It can be appreciated that these fluorescent labels are presented as examples only. Other fluorescent labels having approximately the same spectral properties can be used without limit.

In some embodiments, the systems of the present disclosure comprise dual labeled probes, where the 5′ end is labeled with fluorophore and 3′ end with a quencher. After selective hybridization to the target sequence (PCR amplicon), the probe is cleaved by the 5′-3′ exo+DNA polymerase and fluorescence is subsequently released. In some embodiments, the probe is also quenched at internal bases.

In some embodiments, these probe assays use two sequence-specific oligonucleotide probes in addition to two sequence-specific DNA primers. In some embodiments, the two probes are designed to bind to adjacent sequences in the target nucleic acids. The probes can be labeled with a pair of dyes that exhibit fluorescence resonance energy transfer (FRET). The donor dye is attached to the 3′ end of the first probe, while the acceptor dye is attached to the 5′ end of the second probe. During real-time PCR, excitation is performed at a wavelength specific to the donor dye, and the reaction is monitored at the emission wavelength of the acceptor dye. At the annealing step, the probes hybridize to their target sequences in a head-to-tail arrangement. This annealing brings the donor and acceptor dyes into proximity, allowing FRET to occur, resulting in fluorescent emission by the acceptor. The increasing amount of acceptor fluorescence is proportional to the amount of PCR product present.

In exemplary embodiments, the systems of the present disclosure can include primer pairs and dual labeled probes configured to target a region in the SARS-CoV-2 EndoRNAse gene, the SARS-CoV-2 Spike gene, and the human RNAse P gene, where the SARS-CoV-2 EndoRNAse gene is labeled with FAM, the SARS-CoV-2 Spike gene is labeled with HEX, and the human RNAse P gene is labeled with Cy5.

In exemplary embodiments, the systems of the present disclosure can include primer pairs and dual labeled probes configured to target one or more target nucleic acids and one or more human internal controls. In other exemplary embodiments, the systems of the present disclosure can include primer pairs and dual labeled probes configured to target one or more target nucleic acids and one or more external controls. In exemplary embodiments, the systems of the present disclosure can include primer pairs and dual labeled probes configured to target one or more target nucleic acids and both internal and external controls. In some embodiments, the external RNA or DNA control are added at a known concentration to each sample prior to analysis to control degradation of RNA, interference with the reverse transcriptase reaction, and/or interference with the PCR reaction.

In one exemplary embodiment, the systems provided herein can include one or more primers and/or one or more dual labeled probes configured to target a region in the SARS-CoV-2 EndoRNAse gene, the SARS-CoV-2 Spike gene, and the human RNAse P gene, and where the SARS-CoV-2 EndoRNAse gene is labeled with FAM, the SARS-CoV-2 Spike gene is labeled with HEX, and the human RNAse P gene is labeled with Cy5.

In another exemplary embodiment, the systems provided herein can include one or more primers and/or one or more dual labeled probes configured to target a region in the SARS-CoV-2 EndoRNAse gene, the SARS-CoV-2 Spike gene, and the external artificial RNA control, and where the SARS-CoV-2 EndoRNAse gene is labeled with FAM, the SARS-CoV-2 Spike gene is labeled with HEX, and the external artificial RNA control is labeled with Cy5.

In yet another exemplary embodiment, the systems provided herein can include one or more primers and/or one or more dual labeled probes configured to target a region in the SARS-CoV-2 EndoRNAse gene, the SARS-CoV-2 Spike gene, the human RNAse P gene, and the external artificial RNA control, and where the SARS-CoV-2 EndoRNAse gene is labeled with FAM, the SARS-CoV-2 Spike gene is labeled with HEX, the human RNAse P gene is labeled with Texas Red and the external control is labeled with Cy5.

In some embodiments, the system can further include one or more primers and/or one or more dual labeled probes configured to target a region in the SARS-CoV-2 EndoRNAse gene, the SARS-CoV-2 Spike gene, and the external artificial RNA control, and where the SARS-CoV-2 EndoRNAse gene is labeled with FAM, the SARS-CoV-2 Spike gene is labeled with FAM, and the external artificial RNA control is labeled with HEX.

In some embodiments, the system can further include one or more primers and/or one or more dual labeled probes configured to target a region in the SARS-CoV-2 EndoRNAse gene, the SARS-CoV-2 Spike gene, and the external artificial RNA control, and where the SARS-CoV-2 EndoRNAse gene is labeled with FAM, the SARS-CoV-2 Spike gene is labeled with FAM, and the human RNAse P control is labeled with HEX.

In some embodiments, the system further can further include one or more primers and/or one or more dual labeled probes configured to target a region in the SARS-Cov-2 EndoRNAse gene, the SARS-CoV-2 Spike gene, Influenza A genome, Influenza B genome and the human RNAse P gene, and where the SARS-CoV-2 EndoRNAse gene is labeled with FAM, the SARS-CoV-2 Spike gene is labeled with FAM, Influenza A genome is labeled with HEX, Influenza B genome is labeled with Texas Red and the human RNAse P gene is labeled with Cy5.

In some embodiments, the system can further include one or more primers and/or one or more dual labeled probes configured to target a region in the SARS-Cov-2 EndoRNAse gene, the SARS-CoV-2 Spike gene, Influenza A genome, Influenza B genome and the external artificial RNA control, and where the SARS-CoV-2 EndoRNAse gene is labeled with FAM, the SARS-CoV-2 Spike gene is labeled with FAM, Influenza A genome is labeled with HEX, Influenza B genome is labeled with Texas Red and the external artificial RNA control is labeled with Cy5.

In some embodiments, the system can further include one or more primers and/or one or more dual labeled probes configured to target a region in the SARS-Cov-2 EndoRNAse gene, the SARS-CoV-2 Spike gene, Influenza A genome, Influenza B genome, RSV A genome, RSV B genome and the human RNAse P gene, and where the SARS-CoV-2 EndoRNAse gene is labeled with FAM, the SARS-CoV-2 Spike gene is labeled with FAM, Influenza A genome is labeled with HEX, Influenza B genome is labeled with Texas Red, RSV A genome is labeled with Cy5.5 and RSV B genome is labeled with Cy5.5 and the human RNAse P gene is labeled with Cy5.

In some embodiments, the system can further include one or one or more primers and/or one or more dual labeled probes configured to target a region in the SARS-Cov-2 EndoRNAse gene, the SARS-CoV-2 Spike gene, Influenza A genome, Influenza B genome, RSV A genome, RSV B genome and the external artificial RNA control, and where the SARS-CoV-2 EndoRNAse gene is labeled with FAM, the SARS-CoV-2 Spike gene is labeled with FAM, Influenza A genome is labeled with HEX, Influenza B genome is labeled with Texas Red, RSV A genome is labeled with Cy5.5 and RSV B genome is labeled with Cy5.5 and the external artificial RNA control is labeled with Cy5.

In certain exemplary embodiments, the system can include a system for PCR (e.g., RT-PCR), where the system includes a buffer of Tris at a concentration of about 10 to about 100 mM, salts (potassium chloride at a concentration of about 50 to about 100 mM and magnesium chloride at a concentration of about 2 to about 5 mM), a mixture of dNTPs (dATP, dCTP, dTTP, dGTP) at a concentration of about 0.5 to about 0.5 mM each. In some embodiments, the system for PCR further comprises a Taq polymerase. In some embodiments, the Taq polymerase is a thermostable Taq polymerase with 5′-3′ exonuclease activity at a concentration of about 0.02 to about 2 U/uL. In some embodiments, the concentration of the thermostable Taq polymerase is higher than about 2 U/uL. In certain embodiments, the thermostable Taq polymerase is hot started with an aptamer at concentration ranging from about 10 to about 200 nM. In some embodiments, the system for PCR can include a thermostable reverse transcriptase at a concentration of about 0.05 to about 0.5 U/uL. In some embodiments, the concentration of the thermostable reverse transcriptase is higher than about 0.5 U/uL. In some embodiments, the system for PCR can include a nonionic detergent at a concentration of about 0.1% to about 1.0%. In certain embodiments, the nonionic detergent can include Tween 20, NP-40, and/or Triton X-100. In some embodiments, the system for PCR can further include an RNAse inhibitor at a concentration of about 0.05 U/μL to about 0.5 U/μL. In some embodiments, the concentration of RNAse inhibitor is higher than about 0.5 U/μL. In some embodiments, the system for PCR can also include a reducing agent (e.g., DTT) at a concentration higher than about 0.01 mM. In some embodiments, the concentration of the reducing agent (e.g., DTT) is within the range from about 0.1 to about 1.0 mM. In a particular embodiment, the reducing agent and the RNAse inhibitor are added simultaneously. In some embodiments, the system for PCR can further include an RNA carrier at a concentration of about 0.005 to about 0.01 mg/mL. In one exemplary embodiment, the RNA carrier is polyinosinic acid.

In certain exemplary embodiments, the system for PCR can include a buffer, a salt, a mixture of dNTPs, a Taq polymerase, and one or more of following: a thermostable reverse transcriptase, an RNAse inhibitor, and a nonionic detergent, at concentrations disclosed herein. In one exemplary embodiment, the system for PCR can include a buffer, a salt, a mixture of dNTPs, a Taq polymerase, and a nonionic detergent at concentrations disclosed herein. In another exemplary embodiment, the system for PCR can include a buffer, a salt, a mixture of dNTPs, a Taq polymerase, a thermostable reverse transcriptase, and a nonionic detergent at concentrations disclosed herein. In another exemplary embodiment, the system for PCR can include a buffer, a salt, a mixture of dNTPs, a Taq polymerase, an RNAse inhibitor, and a nonionic detergent at concentrations disclosed herein. In yet another exemplary embodiment, the system for PCR can include a buffer, a salt, a mixture of dNTPs, a Taq polymerase, a thermostable reverse transcriptase, an RNAse inhibitor, and a nonionic detergent at concentrations disclosed herein. In other exemplary embodiments, the system for PCR further can include an RNA carrier at concentrations disclosed herein.

In other exemplary embodiments, the system for PCR can include a buffer, a salt, a mixture of dNTPs, a Taq polymerase, and one or more of following: a thermostable reverse transcriptase, an RNAse inhibitor, and a reducing agent at concentrations disclosed herein. In one exemplary embodiment, the system for PCR can include a buffer, a salt, a mixture of dNTPs, a Taq polymerase, and a reducing agent at concentrations disclosed herein. In another exemplary embodiment, the system for PCR can include a buffer, a salt, a mixture of dNTPs, a Taq polymerase, a thermostable reverse transcriptase, and a reducing agent at concentrations disclosed herein. In one exemplary embodiment, the system for PCR can include a buffer, a salt, a mixture of dNTPs, a Taq polymerase, an RNAse inhibitor, and a reducing agent at concentrations disclosed herein. In another exemplary embodiment, the system for PCR can include a buffer, a salt, a mixture of dNTPs, a Taq polymerase, a thermostable reverse transcriptase, an RNAse inhibitor, and a reducing agent at concentrations disclosed herein. In other exemplary embodiments, the system for PCR can further include an RNA carrier at concentrations disclosed herein. In a particular embodiment, the reducing agent and the RNAse inhibitor are added simultaneously.

In one exemplary embodiment, the system for PCR can include a buffer, at least one salt, a mixture of dNTPs, a thermo stable reverse transcriptase, a Taq polymerase, RNAse inhibitors, a reducing agent, a nonionic detergent, and an RNA carrier at concentrations disclosed herein. In one particular embodiment, the system for PCR can include Tris, KCl, MgCl₂, a mixture of dNTPs, thermostable reverse transcriptase, Taq polymerase, RNAse inhibitors, DTT, Tween 20, RNA carrier at concentrations disclosed herein.

In certain embodiments, the system for PCR has a pH of about 8.4 to about 8.6. In further embodiments, the system for PCR can also include primers and dual labeled probes for viral EndoRNAse (FAM), viral Spike (HEX), and human RNAse P (Cy5).

### Kits

The present disclosure also encompasses kits. A “kit” can include a package, or the like, including some or all of the components of the systems provided herein. In some embodiments, kits include additional components that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains a plurality of primers.

In some embodiments, the kits can include the RT-PCR mixture, the primers and probes. In some embodiments, the target nucleic acids are included in the RT-PCR mixture. In some embodiments, the target nucleic acids are provided separately. In some embodiments, the kits include the RT-PCR mixture provided in a concentrate.

In some embodiments, the kits further include at least one of a control sample, PCR grade water, or combinations thereof. In some embodiments, the control sample can be one or more control samples. The one or more control samples can include a positive control sample, a negative control sample, or both. In some embodiments, the positive control sample comprises either target RNA or DNA in a preserving solution. In some embodiments, in case of viral nucleic acid detection, the positive control comprises heat-inactivated virus. In some embodiments, the negative control sample comprises water or a sample without target nucleic acids. In some embodiments, the kits further comprise at least one of an external DNA control, an RNA control, or both. In some embodiments, the kits further include one or more instructions.

### Methods

The present disclosure also encompasses methods for detecting a target nucleic acid derived from a biological sample. In some embodiments, the methods include one or more of the following steps: (1) contacting the biological sample with the systems provided herein, and (2) subjecting the biological sample and the system to PCR.

A polymerase chain reaction (PCR) generally refers to an in vitro amplification reaction of polynucleotide sequences by the simultaneous primer extension of complementary strands of a double stranded polynucleotide. PCR reactions produce copies of a template polynucleotide flanked by primer binding sites. The result, with two primers, is an exponential increase in template polynucleotide copy number of both strands with each cycle, because with each cycle both strands are replicated. The polynucleotide duplex has termini corresponding to the ends of primers used. PCR can include one or more repetitions of denaturing a template polynucleotide, annealing primers to primer binding sites, and extending the primers by a DNA or RNA polymerase in the presence of nucleotides. Particular temperatures, durations at each step, and rates of change between steps depend on many factors well-known to those of ordinary skill in the art.

In some embodiments, PCR comprises reverse transcription PCR (RT-PCR), quantitative (real-time) PCR (qPCR), nested PCR, quantitative reverse transcription PCR (RT-qPCR), multiplexed PCR, loop-mediated isothermal amplification (LAMP), RT-LAMP, or any combination thereof.

In some embodiments, the PCR is RT-PCR. RT-PCR generally refers to a PCR reaction preceded by a reverse transcription reaction and a resulting cDNA is amplified. In certain embodiments, the RT-PCR can include, but is not limited to, quantitative reverse transcription PCR (RT-qPCR), multiplexed PCR, loop-mediated isothermal amplification (LAMP), RT-LAMP, or any combination thereof, as discussed below. In some embodiments, the reverse transcription reaction and the PCR are conducted simultaneously. In some embodiments, the reverse transcription reaction and the PCR are conducted separately.

In some embodiments, the PCR is a nested PCR. Nested PCR generally refers to a two-stage PCR wherein an amplicon of a first PCR reaction using a first set of primers becomes the sample for a second PCR reaction using a second primer set, at least one of which binds to an interior location of an amplicon of a first PCR reaction.

In some embodiments, the PCR is a real-time PCR. Real-time PCR generally refers to a PCR reaction that monitors the amplification of a targeted DNA molecule during the PCR (e.g., in real time) rather than at the end of the PCR. Real-time PCR can be used quantitatively and semi-quantitatively. Real-time PCR is typically carried out in a thermal cycler with the capacity to illuminate each sample with a beam of light of at least one specified wavelength and detect the fluorescence emitted by an excited fluorophore.

In some embodiments, the PCR is a multiplexed PCR. Multiplexed PCR generally refers to a PCR method where a plurality of polynucleotide sequences is subjected to PCR in the same reaction mixture simultaneously.

In some embodiments, the PCR is a quantitative PCR. Quantitative PCR comprises a PCR reaction designed to measure an absolute or relative amount, abundance, or concentration of one or more sequences in a sample. Quantitative measurements can include comparing one or more reference sequences or standards to a polynucleotide sequence of interest.

In some embodiments, the PCR can include real-time PCR, reverse transcription PCR, quantitative PCR, or any combination thereof.

In some embodiments, the PCR comprises a LAMP or a RT-LAMP. Loop-mediated isothermal amplification (LAMP) is known in the art and generally refers to an isothermal nucleic acid amplification technique. In contrast to other PCR technologies, in which the reaction is carried out with a series of alternating temperature steps or cycles, isothermal amplification is carried out at a constant temperature, and does not require a thermal cycler. Reverse Transcription Loop-mediated Isothermal Amplification (RT-LAMP) combines LAMP with a reverse transcription step to allow the detection of RNA. In LAMP, the target sequence is typically amplified at a constant temperature of about 60° C. to about 65° C. using either two or three sets of primers and a polymerase with high strand displacement activity in addition to a replication activity.

One distinguishing feature of embodiments of the present disclosure is that the systems and methods provided here do not require extracting the target nucleic acid from the biological sample. This feature render the present disclosure a uniquely advantageous option for testing large populations in a pandemic time, when health care specialists have to handle large amount samples patented infected with deadly pathogens, such Corona viruses.

In other embodiments, the systems and methods provided herein do not require pretreatment of the biological samples. However, the systems and methods provided herein are compatible with various pretreated biological samples. Since these pretreatment methods are commonly used in the field, this renders the systems and methods provided herein useful with many sources of the samples.

Thus, in some embodiments, the biological sample is not pretreated. In other embodiments, the biological sample is pretreated. In other embodiments, the biological sample is pretreated at about 65° C. to about 95° C. for about 10 to about 60 minutes. In further embodiments, the biological sample is centrifuged. In some exemplary embodiments, the biological sample is pretreated with heat, proteinase K, or both.

Various sample pretreatments have been tested, which render the sample noninfectious prior opening the tube. In some embodiments, these pretreatments make sample handling safer to the laboratory workers. In case the results need to be obtained quickly and/or the samples are analyzed in an automated way, pretreatments can be skipped for most of the samples, which makes the process extremely simple and fast. In some embodiments, test results can be obtained in under 1 hour.

In some embodiments, the biological sample can be saliva. In certain embodiments, saliva can be used without any pretreatment without loss of sensitivity. In other embodiments, saliva can be pretreated by heat, proteinase K, or both. As an example, pretreatment by heat can include heating to a temperature within the range from about 65° C. to about 95° C. for about 10 to about 60 minutes. In further embodiments, saliva can be centrifuged.

In some embodiments, blood serum, blood EDTA plasma, blood citrate plasma, and blood heparin plasma can be used without any pretreatment without any loss of sensitivity. In some embodiments, the samples are typically handled in an automated way as described herein, so no pretreatment is necessary.

In some embodiments, viral transport media, such as Copan universal (viral) transport medium or PBS can be used without any pretreatments. In some embodiments, the viral transport media can be added in a volume of up to about 5 μL into about 15 μL of the systems provided herein without losing sensitivity of viral RNA detection. In some embodiments, the viral transport media consists up to 25% of the final reaction volume).

Heat inactivation can be included in the methods provided herein whenever safety concerns arise during the handling of the samples. In some embodiments, a heat inactivation step makes the methods safer. For example, in some embodiments, the biological sample is a swab vial with the swab stick in the media and the sticks are manually discarded. In such embodiments, a heat inactivation step can be included. In some embodiments, the heat inactivation involves heating the sample at about 65° C. to about 80° C. for 10 to 30 minutes. It is confirmed that the heating under such conditions does not change the detected amount of the viral RNA. In some embodiments, the heat inactivation can be done in an incubator prior first opening the vial. Some examples are provided in Example 5.

In some embodiments, the biological sample is a pooled sample. A pooled sample typically comprises samples from more than one individual.

In some embodiments, the methods provided herein, such as a RT-PCR viral detection, work with multiplex detection (e.g. two viral genes in FAM and HEX channels, internal RNAse P control or external RNA control in Cy5 channel) with common RT-PCR protocols. In an exemplary embodiment, the methods provided herein include a reverse transcriptase step for about 10 min, which can be any length between 2 to 30 minutes, at about 50° C., which can be any temperature between about 37° C. to about 60° C., followed by denaturation for 2 min at 95° C., then 45 cycles of PCR. An exemplary PCR cycler program includes: 5 seconds at 95° C., followed by 15 seconds at 60° C., and followed by 15 seconds at 72° C. However, the PCR cycling protocol can be shortened to include, for example, 45 cycles of 1 second at 95° C., followed by 5 seconds at 60° C., and followed by 1 second at 72° C., which makes is possible to run the full RT-PCR protocol in just 50 minutes on the most widely used PCR cyclers (Biorad, Roche). One skilled in the art would know how to optimize the PCR. Together with completely skipping the RNA isolation, the methods provided herein make possible to receive the result in only one hour.

The volume of PCR reactions can be anywhere from about 20 pL to 1000 μL. In some embodiments, a typical reaction volume of is about 5 μL. In some embodiments, a typical reaction volume of is about 10 μL. In some embodiments, a typical reaction volume of is about 15 μL. In some embodiments, a typical reaction volume of is about 20 μL. In some embodiments, a typical reaction volume of is about 25 μL. In some embodiments, a typical reaction volume of is about 30 μL. In some embodiments, a typical reaction volume of is about 40 μL. In some embodiments, a typical reaction volume of is about 50 μL.

In some embodiments, the RT-PCR mixture is provided as ready to use concentrate, enabling addition of up to 5 μL sample. In some embodiments, if less sample is added, the rest volume can be supplied with PCR grade water.

In some embodiments, internal RNA controls (such as RNAse P transcript) are used in the samples. Some advantages of internal control is that it shows the quality of the sample and it controls the successful addition of the sample and its simplicity, especially beneficial in automation. In such embodiments, no external control is needed. However, the internal control provides only limited information about the RT-PCR efficacy, because the amount of the internal control can vary among the samples.

In some embodiments, external RNA control is added to the reaction after addition of the sample. In some embodiments, the external RNA control provides exact information about potential RNA degradation and/or RT-PCR efficacy for the particular sample. In other embodiments, the external RNA control provides exact information during development and validation of the RT-PCR mixture. the external RNA control. In some embodiments both the internal and external controls are detected.

It is understood that all described features of the systems and methods can be applied also for the detection of DNA in a sample using PCR.

In some embodiments, RNA viruses can be detected directly in native saliva with the systems and methods described herein. In some embodiments, the method is referred to as direct RT-PCR as it directly detects the presence and/or quantity of target nucleic acids, such as viral RNA, from a biological sample without the need to extract the target nucleic acids. However liquid handling of native saliva can be difficult, especially in automated settings (they are viscous). Thus, in some embodiments, the methods comprise a heat inactivation step as described herein. In some exemplary embodiments, the heating comprises about 10 minutes at 65° C., which is the minimum recommended inactivation to kill the SARS-Cov-2 virus. In some embodiments, the heat inactivation also lowers the viscosity of the sample and makes the sample handling, such as pipetting, easier. In some embodiments, the inactivation makes the detection more robust and increases the sensitivity of virus in some individuals by up to several fold (up to 10-fold in most difficult samples). In some embodiments, temperatures between 65 and 95° C. and times ranging from 2 minutes to 60 minutes or more can be used for sensitive detection. In some embodiments, the most robust results are achieved at 80° C., where short time of several minutes can be used in regular incubators. Alternatively, the heat inactivation can be done in dry bath or PCR cycler. In contrast to other protocols, where at least 30 minutes at 95° C. is necessary, which can be hard to achieve in a regular incubator and one more sample transfer step is needed to transfer to PCR plastic to enable inactivation in PCR cycler, the methods provided herein require much shorter inactivation. In other embodiment, the methods provided herein do not require heat inactivation at all.

In some embodiments, the sensitivity of direct RT-PCR performed using the systems and methods provided herein is significantly improved as compared to other RT-PCR methods that skip the RNA extraction. The sensitivity of the RT-PCR detection performed using the systems and methods described herein is the same in viral transport media (Examples 2, 3 and 4) as with the RNA extraction method, but is significantly more sensitive when used for detection in saliva as compared to conventional RNA extraction protocols followed by conventional RT-PCR (see Example 5).

Some RT-PCR samples can create precipitate, which should be briefly centrifuged at low speed to prevent clogging of tips used in automated liquid handling. At the same time, high speed centrifugation should be avoided, as it lowers the sensitivity of detection in some samples.

In some embodiments, the systems and methods described herein are compatible with samples that have been treated with proteinase K. In some embodiments, only heat inactivation of the proteinase is necessary prior addition of the sample to the systems provided herein. In certain exemplary embodiments, the heat inactivation of the proteinase is performed at about 80° C. or higher for about 10 minutes. In certain exemplary embodiments, the heat inactivation of the proteinase can be performed at a temperature up to about 95° C.

In some embodiments, viral RNA (likely present inside the intact viral particles) survives at least one week at ambient temperature or refrigerated temperature (e.g. in the range of approximately 5° C. to 25° C.). This simplifies the sample collection and transportation. In these cases, no stabilizing solution is needed.

### Automated Methods

The ease of the sample pretreatment (requiring no pretreatment or only heat inactivation prior to opening the vial) makes the direct RT-PCR methods described herein suitable for high throughput automation of the diagnostic process. In some embodiments, using the RT-PCR methods described herein in combination with saliva collection tubes configured to fit into the 96-well SBS format enables processing of tens of thousands of samples using simple equipment and PCR thermocyclers. In some embodiments, the collection tubes can be coated with proteinase K to make the liquid handling more robust.

In some embodiments, the present disclosure provides automated methods for detecting target nucleic acids. In some embodiments, the automated method includes contacting a saliva sample from a subject with the systems described herein, where the saliva sample has been collected in an automation-compatible sample collection device. In some embodiments, the automated method further comprises subjecting the saliva sample directly to real-time RT-PCR. In some embodiments, the automated method comprises collecting the saliva sample from the subject by positioning the automation compatible sample collection device. In some embodiments, the sample collection device comprises: (a) a hollow upper portion that tapers between a first open end and a second open end, the upper portion defining a sample collection volume; (b) a tubular lower portion extending along a longitudinal axis from the second open end to an open terminal end of the device, the lower portion defining a lumen in fluid communication with the sample collection volume and the open terminal end; and (c) at least one groove, extending inward from an outer surface of the lower portion, and extending along a portion of the length of the lower portion. In some embodiments, the at least one groove may be a plurality of circumferentially spaced grooves. In some embodiments, the at least one groove extends along approximately the entire length of the lower portion. In some embodiments, the at least one groove is approximately parallel to the longitudinal axis. In some embodiments, the depth of the groove in the radial direction is between about 0.1 mm and about 1.0 mm. In some embodiments, the terminal end of the lower portion is beveled.

In some embodiments, collecting the saliva sample from the subject further comprises receiving the saliva sample in the sample collection volume, receiving at least a portion of the saliva sample in a sample tube coupled to the sample collection device, and decoupling the sample tube from the sample collection device after receiving the saliva sample. In some embodiments, the saliva collection method further comprises sealing the sample tube after decoupling the sample tube from the sample collection devices.

In some embodiments, collecting the saliva sample from the subject further comprises coupling the sample collection device to the sample tube prior to receiving the saliva sample. For example, coupling the sample collection device to the sample tube may include engaging first threads positioned on an outer surface of the upper portion at about the second end with mating second threads positioned on an inner surface of the sample tube. In some embodiments, the terminal end of the lower portion is distanced between about 5 mm and about 10 mm from a base of the sample tube when the sample collection device is coupled to the sample tube. In some embodiments, an outer diameter of the lower portion is approximately equal to an inner diameter of the sample tube. In some embodiments, a total volume of the sample tube is within the range from about 0.5 mL to about 2.0 mL. In some embodiments, the sample collection device and/or the sample collection device may be coated with a chemical substance. For example, the chemical substance may be a chelating agent, a detergent, a protease, or chaotropic salts.

### EXAMPLES

### Example 1: Detection of SARS-Cov-2 Viral RNA

This Example shows the systems and methods provided herein are ultra-sensitivity in detecting target nucleic acids.

In this Example, RNA was isolated from a SARS-Cov-2 viral culture and quantified against a standard of purified RNA with known concentration. This viral RNA was diluted in a series to concentrations of 20,000, 2,000, 200, 20, 10, 4, and 2 copies per μL (e.g., 100,000; 1,000; 100; 50; 20; 10 copies per 5 μL reaction) and used as samples in RT-PCR reactions.

RT-PCR mixture was prepared as described earlier, which contained Tris buffer, KCl, MgCl₂, dNTPs, reverse transcriptase, Taq polymerase, RNAse inhibitors, DTT, Tween 20, RNA carrier, at the concentrations indicated in the description.

Prior to detection, 5 μL of the viral RNA was combined with 15 μL of the 1.33-fold concentrate of the RT-PCR mix containing primers and dual labeled probes for viral EndoRNAse (FAM), viral Spike (HEX) and human RNAse P (Cy5). After transferring the sample to PCR plate and mixing, the plate was sealed with an optical sealing foil and viral genes and RNAse P were amplified using RT-PCR protocol consisting of 10 min at 50° C., followed by denaturation 2 min 95° C. and then 45 cycles of PCR: 5 sec 95° C.+15 sec 60° C.+15 sec 72° C. in Roche Lightcycler® 480 II instrument. In each cycle, fluorescence in FAM, HEX, and Cy5 was acquired and Ct values were calculated using threshold method.

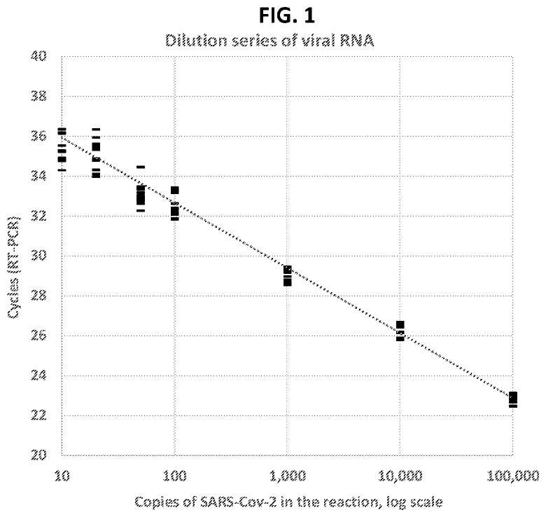

Each concentration was tested in twelve replicates. As shown in FIG. 1, viral RNA was detected linearly in the complete tested range. Each concentration including the lowest concentration of 2 copies per μL (10 copies per reaction) were successfully detected in all 12 replicates. This direct RT-PCR method provided herein can detect single copy of the viral RNA. This example demonstrates that the describe formulation of the RT-PCR mixture is capable of ultrasensitive and robust viral RNA detection.

### Example 2: Detection of SARS-Cov-2 Virus Spiked into Various Biological Samples

This Example shows that the systems and methods provided herein can be used to detect target nucleic acids in various biological samples.

SARS-Cov-2 viral culture was quantified against a standard of known concentration (more precisely, RNA was extracted and then compared to known standard, data were extrapolated for the original culture used to spike the biological samples).

This viral culture was used to spike various biological matrices: buffer, pooled human blood serum, Copan universal transport medium, untreated pooled saliva, heat inactivated saliva, at 65° C. and 80° C. for 10 minutes and the virus was quantified by the RT-PCR. The protocol was the same as in example 1, where 5 μL of the sample were combined with 15 μL of the 1.33-fold concentrate of the RT-PCR mixture and then analyzed in PCR cycler.

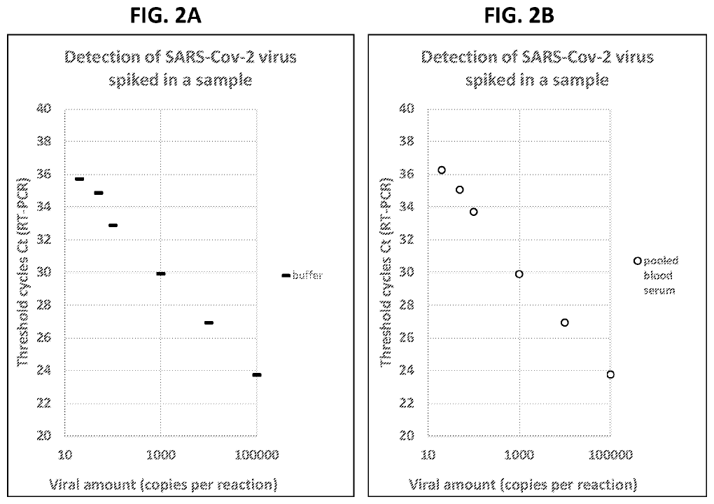

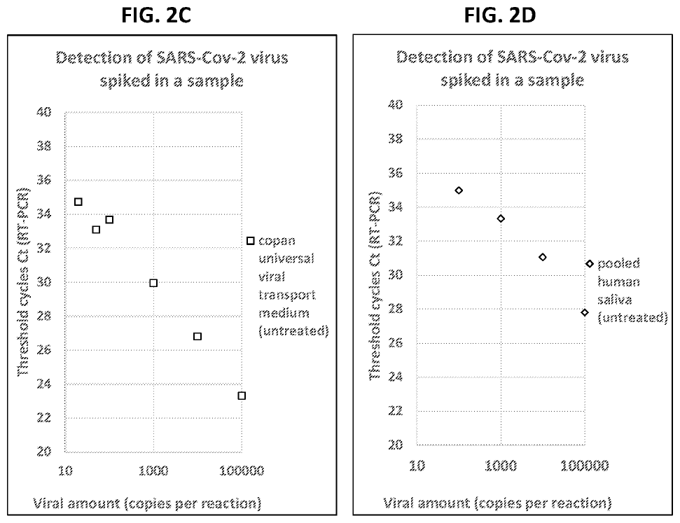

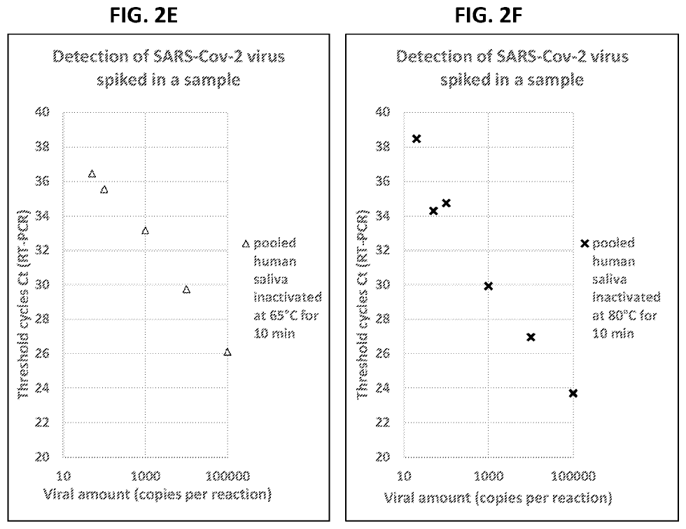

As can be seen on FIG. 2, the lowest concentration of 4 copies per μL (20 copies per 5 μL reaction) of the sample was detected in all samples except non-treated pooled saliva and pooled saliva inactivated at 65° C. for 10 min. There was no significant difference in threshold cycles or sensitivity of detection between buffer, transport medium, blood serum and pooled saliva inactivated at 80° C. for 10 min.

### Example 3: Detection of SARS-Cov-2 Virus in Inactivated Vs Non-Inactivated Clinical Samples of Nasopharyngeal Swabs in Copan Universal Transport Medium

This Example shows that the systems and methods provided herein are comparable with standard method for detecting SARS-Cov-2 RNA from nasopharyngeal swabs in transport medium. Further, this Example demonstrates that the systems and methods provided herein are compatible with heat inactivated samples, which would provide safer sample handling.

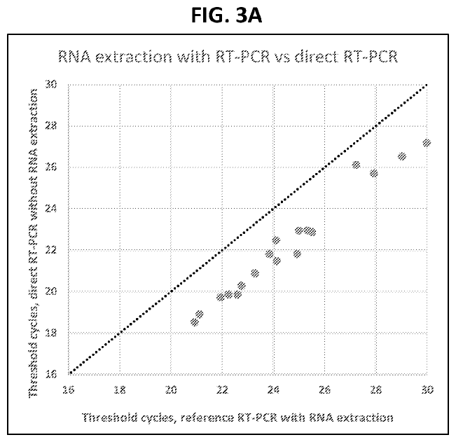

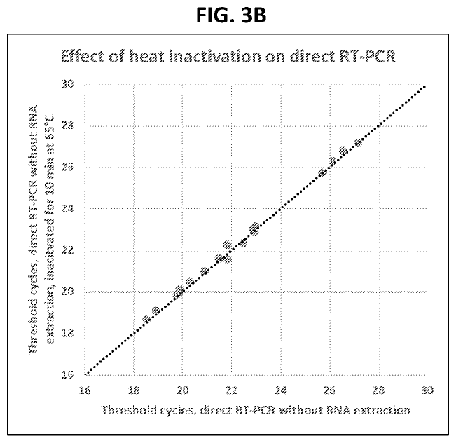

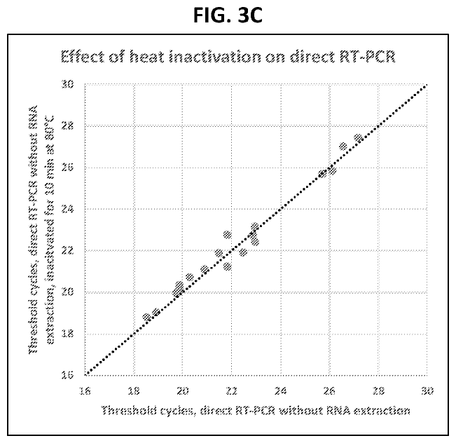

Eighteen samples from SARS-Cov-2 infected individuals were collected and analyzed via standard RNA extraction method on magnetic particles followed by RT-PCR (CE IVD kits from DIANA Biotechnologies were used for RNA isolation and RT-PCR, cat. no. DB-1206 and DB-1211). The same set of samples were retested in direct RT-PCR using the same protocol as in Examples 1 and 2. The threshold cycle values from the standard method and direct RT-PCR were compared. As can be seen, the results from the direct RT-PCR correlated perfectly with the standard method (FIG. 3A). Further, the threshold cycle values from the direct RT-PCR were even lower that the threshold cycle values from the standard method (FIG. 3A).

These results were measured with the native samples. The direct RT-PCR was repeated for the same set of samples after heat inactivation either at 65° C. for 10 min, or at for 10 min. No significant differences to untreated samples were observed (FIGS. 3B and 3C).

### Example 4: Detection of SARS-Cov-2 Virus in Larger Cohort of Clinical Swab Samples

This Example shows that the systems and methods provided herein are comparable with standard method for detecting SARS-Cov-2 RNA from clinical swab samples.

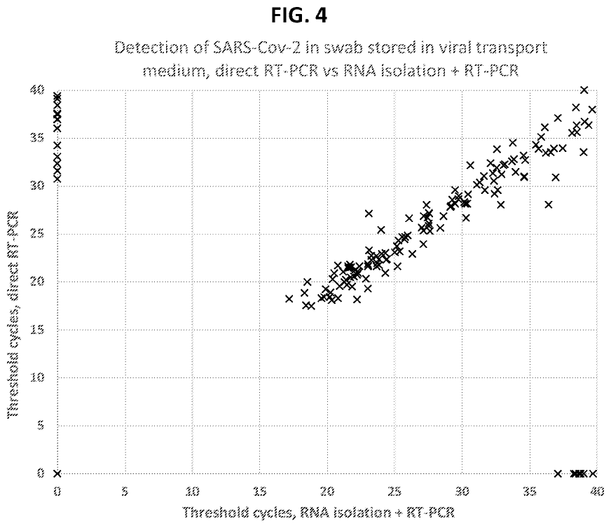

In brief, 537 nasopharyngeal swab samples from individuals suspected of SARS-Cov-2 infection were collected into copan viral transport media and tested in direct RT-PCR in the same way as in previous Examples. They were collected in viral preserving medium and heat inactivated for 10 min at 65° C. prior direct RT-PCR. The results were compared to the standard method of RNA isolation followed by RT-PCR run in an accredited clinical laboratory (FIG. 4).

In total, 148 samples were tested positive in direct RT-PCR. In the standard RNA extraction followed by RT-PCR assay, 142 samples were positive. Fourteen samples were positive only in direct RT-PCR, while eight samples were positive only in the standard protocol, but all were extremely weak over 36^{th} cycle. This shows higher sensitivity of the direct detection over the standard RNA isolation and RT-PCR. This shows that direct RT-PCR from swabs can be used in clinical diagnostics.

### Example 5: Comparison of Detection of SARS-Cov-2 Virus in Saliva by Standard Method of RNA Isolation Followed by RT-PCR Vs Detection by Direct RT-PCR

This example shows that the systems and methods provided herein are more sensitive than standard and more tedious methods for detection of viral RNA in the saliva.

First, to test robustness of the detection with direct RT-PCR from saliva, 8 positive saliva samples were selected and heat inactivated them at different temperatures and times: at 80° C., and 95° C., each for 2, 10, and 30 minutes, respectively. The threshold cycle values for these samples ranged between 20 and 38 cycles, with two very weak samples included. All samples were detected, however the 80° C. inactivation was most robust for the weakest samples, with most positive replicates detected. Similar experiment was repeated for 24 positive saliva samples treated with proteinase K showing that inactivation at 80° C. is suitable as well.

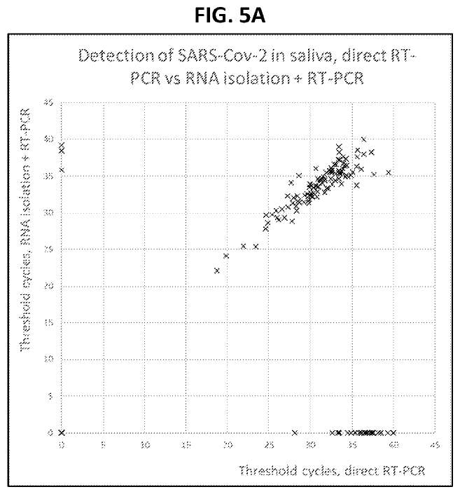

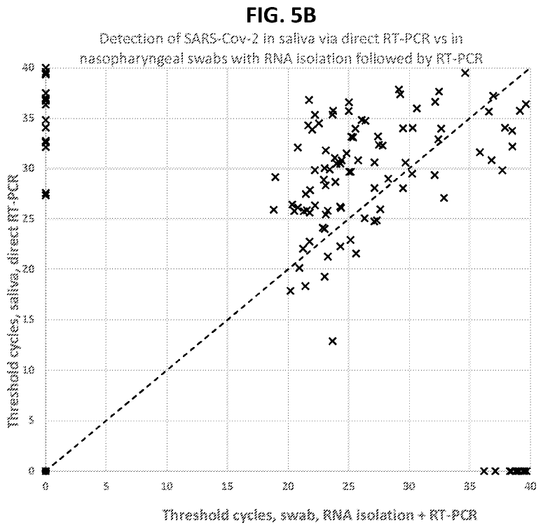

Afterwards, 445 saliva samples from individuals suspected of SARS-Cov-2 infection were collected. There were minor modifications: the saliva samples were inactivated for 20 min at 80° C. and then only 2 μL of the sample was added to the mixture to final 20 μL reaction volume. The results were then compared to SARS-Cov-2 detection from the same samples by standard RNA isolation and RT-PCR. As can be seen from FIG. 5A, 136 samples were positive in the direct RT-PCR assay. In the standard RNA extraction followed by RT-PCR assay, only 108 samples were positive (79%). This shows that the detection by direct RT-PCR is more sensitive than the regular extraction of RNA, which is surprising.

For each sample, external RNA control was added to the mix, which revealed that the RT-PCR was not inhibited, and RNA was not degraded in any of the samples. This shows that the detection is robust and internal control can be used. Samples were retested with the internal control.

### Example 6: Detection of SARS-Cov-2 Virus in Larger Cohort of Clinical Saliva Samples Compared to SARS-Cov-2 Detection in the Paired Swab Samples

This Example shows that the systems and methods provided herein are advantageous in testing in a pandemic, such as the COVID-19 or influenza pandemic. Specifically, the systems and methods provided herein can be used in self-collection of samples, e.g., saliva samples, which are also much less invasive than the nasopharyngeal swabs. Further, the systems and methods provided herein are compatible with high throughput automation.

494 saliva samples paired with the nasopharyngeal swabs from individuals suspected of SARS-Cov-2 infection were collected. The saliva samples were taken at the same moment as the swab samples and tested in direct RT-PCR in the same way as in previous Examples. There were minor modifications: the saliva samples were inactivated for 20 min at 80° C. and then only 2 μL of the sample was added to the mixture to final 20 μL reaction volume. The results were then compared to SARS-Cov-2 detection from the swab samples by standard RNA isolation and RT-PCR run in accredited laboratory. As can be seen from FIG. 5B, in total 109 saliva samples were positive, while only 105 swab samples were positive. 16 samples were positive only in saliva (including several samples with high viral load) while 12 samples were positive only in swabs, all these samples were very weak (over 35 t h cycle). This show that direct RT-PCR from saliva is even more sensitive than the standard swab linked with standard RNA isolation and RT-PCR. Detection of SARS-Cov-2 from saliva can thus become the new standard.

For each sample, external RNA control was added to the mix, which revealed that the RT-PCR was not inhibited and RNA was not degraded in any of the samples. This shows that the detection is robust and internal control can be used. Samples were retested with the internal control.

Comparing the results from direct RT-PCR from saliva and standard method from nasopharyngeal swabs (Table 1) show that direct RT-PCR from saliva can be used not only for screening the population for infected people but also for diagnostics. Specifically, Table 1 shows that the sensitivity of direct RT-PCR from saliva samples was 104% of the sensitivity of RNA isolation followed by RT-PCR from swab samples.

TABLE 1
Comparison of direct RT-PCR from swab
vs direct RT-PCR from saliva.

Direct RT-PCR from saliva
samples

| | | |
|---|---|---|
| No. of samples | Positive | Negative |

| | | | |
|---|---|---|---|
| RNA iso & | Positive | 93 | 12 |
| RT-PCR from | Negative | 16 | 371 |
| swab samples | | | |

This method will discover more infected people compared to swabs, while it will allow for self collection of the samples, high throughput automation and is much less invasive than the nasopharyngeal swabs. This will be the method of choice to control potential outbreaks in companies and other large populations.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A system for reverse transcriptase polymerase chain reaction (RT-PCR), comprising:
a buffer;
a salt;
a mixture of deoxynucleotide triphosphates (dNTPs);
a detergent;
a reducing agent;
an RNA carrier;
a thermostable DNA polymerase;
a reverse transcriptase; and
an RNAse inhibitor.

2. The system of claim 1, wherein the buffer comprises at least one of Tris, Bis-tris-propane, PIPES, MOPS, or HEPES.

3. The system of any one of the preceding claims, wherein the salt comprises at least one of potassium chloride, ammonium sulfate, magnesium chloride or magnesium sulfate.

4. The system of any one of preceding claims, wherein the buffer further comprises glycerol.

5. The system of any one of preceding claims, wherein the buffer further comprises dimethyl sulfoxide (DMSO).

6. The system of any one of preceding claims, wherein the buffer further comprises Bovine serum albumin (BSA) or casein.

7. The system of any one of preceding claims, wherein the thermostable DNA polymerase comprises at least one of a Taq polymerase, a Tth Polymerase, a Bst polymerase, or a Z05 polymerase.

8. The system of any one of preceding claims, wherein the thermostable DNA polymerase is a wild-type enzyme.

9. The system of any one of preceding claims, wherein the thermostable DNA polymerase comprises one or more single point mutations or is on N-terminus, C-terminus, internally truncated, or fused to another peptide or protein.

10. The system of any one of preceding claims, wherein the RT-PCR comprises at least one of a quantitative reverse transcription PCR (RT-qPCR), a loop-mediated isothermal amplification (LAMP), a RT-LAMP, or any combination thereof.

11. The system of any one of preceding claims, wherein the thermostable DNA polymerase does not have 5′-3′ exonuclease activity.

12. The system of any one of claims 1-10, wherein the thermostable DNA polymerase has exonuclease activity.

13. The system of any one of preceding claims, wherein the buffer comprises Tris at a concentration within the range from about 10 to about 100 mM.

14. The system of any one of preceding claims, wherein the salt comprises potassium chloride at a concentration within the range from about 50 to about 100 mM.

15. The system of any one of preceding claims, wherein the salt comprises magnesium chloride at a concentration of about 1 to about 5 mM.

16. The system of any one of preceding claims, wherein the salt comprises ammonium sulfate at a concentration within the range from about 20 to about 50 mM.

17. The system of any one of preceding claims, wherein the salt comprises magnesium sulfate at a concentration within the range from about 1 to about 5 mM.

18. The system of any one of preceding claims, wherein the mixture of dNTPs comprises dATP, dCTP, dTTP, and dGTP, each at a concentration within the range from about 0.05 to about 0.5 mM.

19. The system of any one of preceding claims, wherein the mixture of dNTPs comprises dATP, dCTP, dUTP, and dGTP, each at a concentration within the range from about 0.05 to about 0.5 mM.

20. The system of any one of preceding claims, wherein the reverse transcriptase is thermostable.

21. The system of any one of preceding claims, wherein the reverse transcriptase comprises M-MLV, AMV, or FeLV reverse transcriptase.

22. The system of any one of claims 1-20, wherein the reverse transcriptase is a wild-type enzyme.

23. The system of any one of claims 1-20, wherein the reverse transcriptase comprises one or more single point mutations or is on N-terminus, C-terminus or internally truncated or fused to another peptide or protein.

24. The system of any one of claims 1-20, wherein the reverse transcriptase is an RNAse H⁻ mutant.

25. The system of any one of claims 1-20, wherein the reverse transcriptase is inactivated by aptamer-oligonucleotides at about room temperature.

26. The system of any one of claims 1-20, wherein the reverse transcriptase is inactivated by aptamer-oligonucleotides at temperatures of up to about 45° C.

27. The system of any one of preceding claims, wherein the concentration of the reverse transcriptase is higher than about 0.5 U/uL.

28. The system of any one of claims 1-26, wherein the concentration of the reverse transcriptase is within the range from about 0.05 to about 0.5 U/uL.

29. The system of any one of the preceding claims, wherein the concentration of the DNA polymerase is higher than about 2 U/uL.

30. The system of any one of claims 1-28, wherein the concentration of the DNA polymerase is within the range from about 0.02 to about 2 U/uL.

31. The system of any one of the preceding claims, wherein the DNA polymerase is inactivated by aptamer-oligonucleotides, anti-DNA polymerase antibodies, or chemical modifications at about room temperature.

32. The system of any one of claims 1-30, wherein the DNA polymerase is inactivated by aptamer-oligonucleotides at temperatures of up to about 55° C.

33. The system of any one of preceding claims, wherein the reducing agent is selected from the list consisting of Dithiothreitol (DTT), (3-mercaptoethanol, tris(2-carboxyethyl)phosphine (TCEP), glutathione, acetyl L-cystein, acetyl D-cystein, L-Cysteine methyl ester, D-Cysteine methyl ester, L-Cysteine methyl ester, D-Cysteine methyl ester, N-Formyl-L-cysteine, Tris(hydroxypropyl)phosphine, Tris(hydroxymethyl)phosphine, Sodium triacetoxyborohydride, 1,2-Ethanedithiol, 2-Mercaptopropan-1-ol, 3-Mercaptopropan-1-ol, 1-mercaptopropan-2-ol, Thioglycolic acid and a salt, Dithiothreitol, 2-Mercaptobenzoic acid, 3-Mercaptobenzoic acid, 4-Mercaptobenzoic acid, 4-Mercaptobutan-1-ol, Cysteamine, homocysteine, N-Acetyl-L-homocysteine, L-homocysteine methyl ester, 3-mercaptobutanol, Dihydrolipoic acid, dithiobutylamine, sodium sulfite, NADH, FADH₂, 2,3-Pyrazinedithiol, thiourea, or thiolactic acid.

34. The system of any one of claims 1-32, wherein the reducing agent is Dithiothreitol (DTT).

35. The system of claim 34, wherein the concentration of DTT is higher than about 0.01 mM.

36. The system of claim 34, wherein the concentration of DTT is within the range from about 0.1 to about 1.0 mM.

37. The system of any one of preceding claims, wherein the RNAse inhibitor remains active at a temperature up to at least about 40° C.

38. The system of any one of preceding claims, wherein the RNAse inhibitor is selected from the list consisting of a porcine liver RNAse inhibitor, a human placental RNAse inhibitor, a murine RNAse inhibitor, a rat lung RNAse inhibitor, or a rat liver RNAse inhibitor.

39. The system of any one of claims 1-37, wherein the RNAse inhibitor comprises one or more single point mutations or is on N-terminus, C-terminus or internally truncated or fused to another peptide or protein.

40. The system of any one of preceding claims, wherein the concentration of the RNAse inhibitor is about 0.1 U/uL or higher.

41. The system of any one of claims 1-39, wherein concentration of RNAse inhibitor is in the range of about 0.01 to about 0.1 U/uL.

42. The system of any one of preceding claims, wherein the concentration of the RNA carrier is within the range from about 0.0005 to about 0.05 mg/mL.

43. The system of any one of claims 1-41, wherein the concentration of the RNA carrier is within the range from about 0.002 to 0.01 mg/mL.

44. The system of any one of preceding claims, wherein the RNA carrier comprises a polyinosinic acid, a polyinosinic-polycytidylic acid, or a polyadenosine.

45. The system of any one of claims 1-43, wherein the RNA carrier comprises a polyinosinic acid.

46. The system of any one of claims 1-43, wherein the RNA carrier comprises a polyadenosine.

47. The system of any one of preceding claims, wherein the system has a pH within the range of about 8.2 to about 8.8.

48. The system of any one of preceding claims, wherein the system has a pH within the range of about 8.4 to about 8.6.

49. The system of any one of preceding claims, wherein the detergent is a nonionic detergent.

50. The system of claim 49, wherein the concentration of the nonionic detergent is within the range from about 0.05% to about 5%.

51. The system of claim 49, wherein the concentration of the nonionic detergent is within the range from about 0.1% to about 2.0%.

52. The system of claim 49, wherein the concentration of the nonionic detergent is within the range from about 0.2% to about 1.0%.

53. The system of claim 49, wherein the concentration of the nonionic detergent is about 0.5%.

54. The system of any one of preceding claims, wherein the nonionic detergent comprises at least one of Tween 20, Tween 40, Tween 80, Nonidet P40, NP-40, Triton™ X-100, C₁₂E₈, or dodecylmaltoside (DDM).

55. The system of any one of preceding claims, further comprising one or more primers.

56. The system of claim 55, wherein the one or more primers are configured to hybridize to a target nucleic acid.

57. The system of claim 56, wherein the target nucleic acid is derived from a biological sample.

58. The system of claim 56, wherein the target nucleic acid comprises RNA or DNA.

59. The system of claim 56, wherein the target nucleic acid comprises a viral RNA or viral DNA, bacterial RNA or bacterial DNA, animal RNA or animal DNA, human RNA or human DNA.

60. The system of claim 56, wherein the target nucleic acid is SARS-Cov-2 RNA, SARS-1 (2003), MERS, influenza A, influenza B, RSV, Hepatitis A, Hepatitis B, Hepatitis C, or HIV.

61. The system of any one of preceding claims, further comprising one or more primers and/or one or more dual labeled probes configured to target a region in at least one of a SARS-CoV-2 EndoRNAse gene, a SARS-CoV-2 Spike gene, a SARS-CoV-2 ORF1ab gene, SARS-CoV-2 RdRP gene, SARS-CoV-2 E gene, or a SARS-CoV-2 N gene.

62. The system of claim 61, further comprising a primer pair and dual labeled probes configured to target a region in a SARS-CoV-2 EndoRNAse gene and a SARS-CoV-2 Spike gene, respectively.

63. The system of any one of claims 1-60, further comprising one or more primers and/or one or more dual labeled probes configured to target a region in an internal human control gene.

64. The system of any of the claims 1-60, further comprising one or more primers and/or one or more dual labeled probe configured to target an external RNA or DNA control.

65. The system of any one of claims 1-60, further comprising one or more primers and/or one or more dual labeled probes configured to target a region in a SARS-CoV-2 EndoRNAse gene, the SARS-CoV-2 Spike gene, and the human RNAse P gene, wherein the SARS-CoV-2 EndoRNAse gene is labeled with FAM, the SARS-CoV-2 Spike gene is labeled with HEX, and the human RNAse P gene is labeled with Cy5.

66. The system of any one of claims 1-60, further comprising one or more primers and/or one or more dual labeled probes configured to target a region in a SARS-CoV-2 EndoRNAse gene, a SARS-CoV-2 Spike gene, and an external artificial RNA control, wherein the SARS-CoV-2 EndoRNAse gene is labeled with FAM, the SARS-CoV-2 Spike gene is labeled with HEX, and the external artificial RNA control is labeled with Cy5.

67. The system of any one of claims 1-60, further comprising one or more primers and dual labeled probes configured to target a region in a SARS-CoV-2 EndoRNAse gene, a SARS-CoV-2 Spike gene, a human RNAse P gene, and an external artificial RNA control, wherein the SARS-CoV-2 EndoRNAse gene is labeled with FAM, the SARS-CoV-2 Spike gene is labeled with HEX, the human RNAse P gene is labeled with Texas Red, and the external control is labeled with Cy5.

68. The system of any one of claims 1-60, further comprising one or more primers and/or one or more dual labeled probes configured to target a region in a SARS-CoV-2 EndoRNAse gene, a SARS-CoV-2 Spike gene, and an external artificial RNA control, and wherein the SARS-CoV-2 EndoRNAse gene is labeled with FAM, the SARS-CoV-2 Spike gene is labeled with FAM, and the external artificial RNA control is labeled with HEX.

69. The system of any one of claims 1-60, further comprising one or more primers and/or one or more dual labeled probes configured to target a region in a SARS-CoV-2 EndoRNAse gene, a SARS-CoV-2 Spike gene, and a human RNAse P gene, wherein the SARS-CoV-2 EndoRNAse gene is labeled with FAM, the SARS-CoV-2 Spike gene is labeled with FAM, and the human RNAse P gene is labeled with HEX.

70. The system of any one of claims 1-60, further comprising one or more primers and/or one or more dual labeled probes configured to target a region in a SARS-Cov-2 EndoRNAse gene, a SARS-CoV-2 Spike gene, an Influenza A genome, an Influenza B genome and a human RNAse P gene, wherein the SARS-CoV-2 EndoRNAse gene is labeled with FAM, the SARS-CoV-2 Spike gene is labeled with FAM, the Influenza A genome is labeled with HEX, the Influenza B genome is labeled with Texas Red, and the human RNAse P gene is labeled with Cy5.

71. The system of any one of claims 1-60, comprising one or more primers and/or one or more dual labeled probes configured to target a region in a SARS-Cov-2 EndoRNAse gene, a SARS-CoV-2 Spike gene, an Influenza A genome, an Influenza B genome and the external artificial RNA control, wherein the SARS-CoV-2 EndoRNAse gene is labeled with FAM, the SARS-CoV-2 Spike gene is labeled with FAM, the Influenza A genome is labeled with HEX, the Influenza B genome is labeled with Texas Red and the external artificial RNA control is labeled with Cy5.

72. The system of any one of claims 1-60, further comprising one or more primers and/or one or more dual labeled probes configured to target a region in a SARS-Cov-2 EndoRNAse gene, a SARS-CoV-2 Spike gene, an Influenza A genome, an Influenza B genome, a RSV A genome, RSV B genome, and a human RNAse P gene, wherein the SARS-CoV-2 EndoRNAse gene is labeled with FAM, the SARS-CoV-2 Spike gene is labeled with FAM, Influenza A genome is labeled with HEX, Influenza B genome is labeled with Texas Red, RSV A genome is labeled with Cy5.5 and RSV B genome is labeled with Cy5.5 and the human RNAse P gene is labeled with Cy5.

73. The system of any one of claims 1-60, comprising one or more primers and/or one or more dual labeled probes configured to target a region in a SARS-Cov-2 EndoRNAse gene, a SARS-CoV-2 Spike gene, an Influenza A genome, an Influenza B genome, an RSV A genome, an RSV B genome and an external artificial RNA control, wherein the SARS-CoV-2 EndoRNAse gene is labeled with FAM, the SARS-CoV-2 Spike gene is labeled with FAM, the Influenza A genome is labeled with HEX, the Influenza B genome is labeled with Texas Red, RSV A genome is labeled with Cy5.5, the RSV B genome is labeled with Cy5.5, and the external artificial RNA control is labeled with Cy5.

74. A kit comprising the system of any one of the preceding claims.

75. The kit of claim 74, further comprising at least one of a control sample, PCR grade water, or combinations thereof.

76. The kit of claim 75, wherein the control sample comprises a positive control sample, a negative control sample, or both.

77. The kit of claim 76, wherein the control sample comprises an external RNA control.

78. The kit of any one of claims 74-77, further comprising an instruction.

79. A method for detecting a target nucleic acid derived from a biological sample, comprising:
contacting the biological sample with the system of any one of preceding claims; and
subjecting the biological sample and the system to RT-PCR.

80. The method of claim 79, wherein the RT-PCR comprises quantitative reverse transcription PCR (RT-qPCR), a reverse transcription loop-mediated isothermal amplification (RT-LAMP), a LAMP or any combination thereof.

81. The method of any one of claims 79-80, wherein the method does not comprise extracting the target nucleic acid from the biological sample.

82. The method of any one of claims 79-81, wherein the biological sample is not pretreated.

83. The method of any one of claims 79-81, wherein the biological sample is pretreated.

84. The method of claim 83, wherein the biological sample is pretreated with at least one of heat or proteinase K.

85. The method of claim 83, wherein the biological sample is pretreated by heating to a temperature within the range from about 65° C. to about 95° C. for about 10 to about 60 minutes.

86. The method of claim 83, wherein the biological sample is centrifuged.

87. The method of any one of claims 79-86, wherein the biological sample is a pooled sample comprising target nucleic acids from multiple subjects.

88. The method of any one of claim 87, wherein the target nucleic acids from the multiple subjects are detected in one reaction.

89. The method of any one of claims 79-88, wherein the biological sample comprises at least one of blood, blood serum, blood plasma, saliva, nasal swab, nasopharyngeal swab, nasal wash, mouth swab, mouth wash, seminal plasma, or urine, or any combination thereof.

90. The method of any one of claims 79-89, wherein the target nucleic acid comprises at least one of DNA or RNA.

91. The method of any one of claims 79-90, further comprising quantifying the target nucleic acid amplified by the RT-PCR.
